(19) United States
(12) Patent Application Publication
(10) Pub. No.: US 2009/0035876 A1
(43) Pub. Date: Feb. 5, 2009

(54) Electrochemical Assay

(75) Inventors: David Edward Williams, Auckland (NZ); Phillip Lowe, Tullibody (GB); Christopher John Slevin, Edinburgh (GB); Anne-Cecile Herve, Plouezoc'h (FR); Stephen John Carlisle, Nr Newport Pagnell (GB); Alan Thomson, Northamptonshire (GB)

Correspondence Address: NIXON & VANDERHYE, PC, 901 NORTH GLEBE ROAD, 11TH FLOOR, ARLINGTON, VA, 22203, US

(73) Assignee: Inverness Medical Switzerland GMBH, Zug (CH)

(21) Appl. No.: 11/887,393

(22) PCT Filed: Mar. 31, 2006
(86) PCT No.: PCT/GB2006/001177

§ 371 Date: Sep. 8, 2008

Related U.S. Application Data
(60) Provisional application No. 60/666,562, filed on Mar. 31, 2005.

(51) Int. Cl. G01N 33/543

(52) U.S. Cl. 436/529; 436/518; 436/528

(57) ABSTRACT

A method of determining the presence or amount of analyte in a fluid sample, which comprises: contacting a fluid sample with a binding reagent that comprises a plurality of cleavable species and wherein said species, when cleaved, are detectable using electrochemical means; separating any binding reagent-analyte complex that forms from the unbound binding reagent; cleaving the cleavable species from the immobilized binding reagent-analyte complex; and detecting the cleaved species using electrochemical means.

### FIELD OF THE INVENTION

The present invention is concerned with a method of determining the presence or amount of analyte in a fluid sample, a binding reagent for use in such a method, the use of such a binding reagent in an immunoassay and a kit for measuring the amount or presence of an analyte in a sample.

### BACKGROUND TO THE INVENTION

Immunoassays for determining the presence or amount of analyte in a fluid sample which rely upon the use of a binding reagent that binds to the analyte of interest are known. In such devices a binding reagent-analyte complex is formed which is then immobilized at a capture site and the presence or amount of analyte is then determined. Such determination can be performed by various methods, for example fluorescence. However, one problem associated with such assays it that they are sometime not very effective at low analyte concentrations. This is because the concentration of the binding reagent-analyte complex will also be low and it can be difficult to determine the presence and/or amounts of low concentrations of such species. It would be beneficial if a method could be developed which was suitable for determining the presence or amount of analyte in a fluid sample which was effective even at low analyte concentrations.

### SUMMARY OF INVENTION

The present inventors have developed a new method of determining the presence or amount of analyte in a fluid sample which enables accurate detection of an analyte even at low analyte concentration levels.

Accordingly, the present invention provides a method of determining the presence or amount of analyte in a fluid sample, which comprises:
(a) contacting a fluid sample with a binding reagent that comprises a plurality of cleavable species and wherein said species, when cleaved, are detectable using electrochemical means;
(b) separating any binding reagent-analyte complex that forms from the unbound binding reagent;
(c) cleaving the cleavable species from the immobilized binding reagent-analyte complex; and
(d) detecting the cleaved species using electrochemical means.

The present invention also provides a binding reagent of the present invention.

The present invention further provides the use in an immunoassay of a binding regent of the present invention.

The present invention additionally provides an assay kit for measuring the amount or presence of an analyte in a sample, comprising;
(a) a binding reagent of the present invention,
(b) a capture phase comprising a support having a reagent which is capable of binding or attaching to a binding-reagent-analyte complex, and;
(c) an electrode capable of detecting the cleavable species, when cleaved, to provide an indication of the presence or amount of analyte present.

Separation of any formed binding reagent-analyte complex from the unbound binding reagent may be carried out by immobilization of the binding reagent-analyte complex.

### DESCRIPTION OF THE FIGURES

FIG. 1 Generalised scheme for electrochemical measurement of UV cleaved electrochemical reporter group.

FIG. 2 A summary of the assay architecture reported. The 20 μm and 400 nm beads meet the requirements stipulated in FIG. 1.

FIG. 3 TLC of the photolysis of the UV-cleavable ferrocene molecule 9 (Concentration 2.18 mM) at various irradiation times.

FIG. 4 Reagents and conditions: a) 0.4 μm latex particle aldehyde modified, Amino dextran, NaBH₃CN (1 M), MES (50 mM, pH 6.0); b) GMBS, DMF, PBS (pH 7.0); c) Deprotected 9, DMF, PBS (pH 7.0).

FIG. 5 CV's: Before irradiation (2 repeats, represented by —-— and . . . ). After 5 minutes of irradiation (2 repeats, represented by the unbroken line and - - - ). 17 μl of sample applied to screen printed electrode (Carbon working and counter electrodes, silver/silver chloride reference electrode).

FIG. 6 CV's: Before irradiation (2 repeats, represented by —-— and . . . ). After 5 minutes of irradiation (2 repeats, represented by the unbroken line and - - - ). 17 μl of sample applied to screen printed electrode (Carbon working and counter electrodes, silver/silver chloride reference electrode).

FIG. 7 CV's: Before irradiation (2 repeats, represented by —-— and . . . ). After 5 minutes of irradiation (2 repeats, represented by the unbroken line and - - - ). 17 μl of sample applied to screen printed electrode (Carbon working and counter electrodes, silver/silver chloride reference electrode).

FIG. 8 Chronoamperometry measurements of variable bead concentrations (1.16E+08, 46600000, 23300000, 11650000, 5825000, 2912500 beads per 17 μL).

FIG. 9 Chronoamperometry scans of variable bead concentrations. Each concentration has been PBS background corrected, i.e. the PBS background scan has been subtracted from each concentration using the subtract disk file/edit data within the Autolab control software.

FIG. 10 Chronoamperometry measurements of the lowest bead concentration (2912500 beads per 17 μL) (the unbroken line) and the PBS control measurement (the broken line). Note the increasing and decreasing current suggesting depletion of the UV cleaved ferrocene molecule.

FIG. 11 Chronoamperometry measurements of known concentrations of The UV cleaved ferrocene molecule. Measurements were made with identical methodology to the investigation summarised in FIG. 8.

FIG. 12 Calibration curve for the UV cleaved ferrocene molecule. Values (i/A) were extracted from the 200 second points from FIG. 11.

FIG. 13 Plot of particle number vs i/A (cleaved FcPEG). Values were extracted from the 200 second points from FIG. 9.

FIG. 14 Plot of FcPEG (cleaved) vs particle number. The values (i/A) from FIG. 3.12 were converted in FcPEG concentration (μM) using FIG. 12.

FIG. 15 Chronoamperometry measurements of UV cleaved ferrocene molecules, 2 repeats of 38 mV (voltage input LED) 6 μL sample in a capillary fill electrode device, represented by the —-— and . . . . The line - - - represents as previous but 22 mV. The unbroken line represents PBS as previous but 38 mV.

FIG. 16 As shown in FIG. 15 but resealed.

FIG. 17 Reagents and conditions: a) 0.4 μm latex particle aldehyde modified, Amino dextran, NaBH₃CN (1 M), MES (50 mM, pH 6.0), b) GMBS, DMF, PBS (pH 7.0); c) Modified 3299, PBS (pH 7.0); d) Deprotected 9, DMF, PBS (pH 7.0)

FIG. 18 Reagents and conditions: a) 0.4 μm latex particles aldehyde modified, Amino dextran, NaBH₃CN (1 M), MES (50 mM, pH 6.0); b) GMBS, DMF, PBS (pH 7.0); c) Deprotected 9 DMF, PBS (pH 7.0), SHPEG₄CO₂H; d) Amino dextran, EDCI, NHS, MES (50 mM, pH 6.0); e) GMES, DMF, PBS (pH 7.0); f) Modified 3299, PBS (pH 7.0)

FIG. 19 Chronoamperometry measurements of TRF beads 400 nm with both antibody and UV cleavable linker. 17 uL of solution applied to electrode (carbon working, counter and silver/silver chloride reference electrode). The line . . . represents the results obtained when the antibody is coupled first followed by the cleavable linker. The unbroken line represents the results obtained when the cleavable linker is coupled first followed by the antibody.

FIG. 20 Chronoamperometry measurements of TRF beads 400 nm with both antibody and UV cleavable linker. 17 uL of solution applied to electrode (carbon working, counter and silver/silver chloride reference electrode. The unbroken line represents TRF beads 400 nm with both antibody and UV cleavable linker, the line - - - represents ½ dilution of TRF beads 400 nm with both antibody and UV cleavable linker and the line represents the PBS control.

FIG. 21 A rescaled chronoamperometry measurement of TRF beads 400 nm with both antibody and UV cleavable linker (from FIG. 19). The LED input voltage was switched from 22 mV to 38 mV at 504 seconds, the change in rate can clearly be observed.

FIG. 22 Reagents and conditions: a) F108-PMPI, deionised H₂O; b) Modified 3468, PBS (pH7.0).

FIG. 23 Chronoamperometry measurements of 0 (unbroken line) and 400 (broken line) mIU hCG standards. A wet hCG assay has been performed prior to running the solution through the microfluidic IMF 3 device which involved the premixing of the hCG standard, 400 nm 3299/UV-cleavable ferrocene compound (UVCFC) and 20 μm 3468 latex beads for approximately 30 minutes.

FIG. 24 As shown in figure but rescaled to emphasise the difference between the 0 and 400 mIU hCG measurements.

FIG. 25 Percentage binding of electrochemical ferrocene compounds to HAS where ferrocene PEG is modified with a 0-12 carbon chain.

FIG. 26 Chronoamperograms of IT17 in PBS at 2 terminal interdigitated electrode. 2 μm line and gap (CSEM carbon electrode).

FIG. 27 Determination of IT17 in PBS at 2 terminal interdigitated electrode. 2 μm line and gap (CSEM carbon electrode).

FIG. 28 Differential pulse, uncoated electrodes. Sensitivity of IT17, various concentrations: the line —-— represents 2.5 μM. The line . . . represents 1 μM. The unbroken line represents 750 nM. The line - - - represents 500 nM. The line——-— represents 250 nM. The line —--— represents PBS.

FIG. 29 Broken line represents PBS. Unbroken line represents 50 μM IT17 in PBS. Potential swept from 0V to 0.4V by 100 mV/s, then held ast 0.4V during 120 s, then scanned back to 0V by 100 mV/s. Electrodes coated with nafion 0.1% cast from EtOH. Scans run 2 min after solutions applied to electrodes.

FIG. 30 Broken line represents PBS. Unbroken line represents 50 μM IT17 in PBS. Potential swept from 0V to 0.4V by 100 mV/s, then held ast 0.4V during 120 s, then scanned back to 0V by 100 mV/s. Electrodes coated with nafion 0.1% cast from H₂O. Scans run 2 min after solutions applied to electrodes.

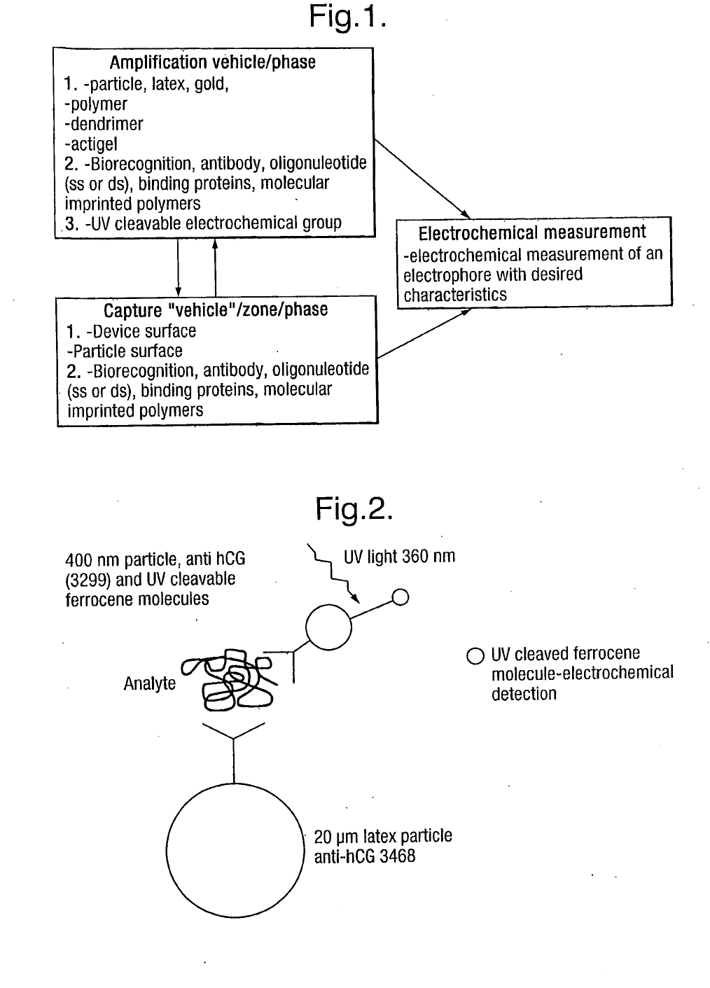

### DETAILED DESCRIPTION OF THE INVENTION

The method of determining the presence or amount of analyte in a fluid sample may be an assay such as a heterogeneous assay, for example a lateral flow or microfluidic type of assay wherein a binding reagent-analyte complex is immobilised at the surface of a capture phase. Once the binding reagent-analyte complex has been immobilised at the capture phase, the cleavable species can be cleaved and then detected using electrochemical means, such means can, for example comprise an electrode or an electrode surface.

Any suitable method can be used to separate the binding reagent-analyte complex from the unbound binding reagent. Filtration is an example of such a method. A further example of a suitable separation method involves the formation of a complex of a magnetically labelled binding reagent and the binding reagent-analyte complex followed by the separation of the binding reagent-analyte-magnetically labelled binding reagent complex from the unbound binding reagent by the use of a magnet. Preferably, the binding reagent-analyte complex and the unbound binding reagent are separated by immobilization of the binding reagent-analyte complex in a capture phase.

The binding reagent for use in the present invention may be chosen from any that is able to bind to the analyte of interest to form a binding pair. Examples of binding pairs include an antibody an antigen, biotin and avidin, polymeric acids and bases, dyes and protein binders, peptides and specific protein binders, enzymes and cofactors, and effector and receptor molecules, where the term receptor refers to any compound or composition capable of recognising a particular or polar orientation of a molecule, namely an epitopic or determinant site.

Reference to an antibody includes but is not limited to, polyclonal, monoclonal, bispecific, humanised and chimeric antibodies, single chain antibodies, Fab fragments and F(ab′)₂ fragments, fragments produced by a Fab expression library, anti-idiotypic (anti-Id) antibodies, and epitope-binding fragments of any of the above. Portions of antibodies include Fv and Fv′ portions.

Thus, the binding reagent will in general comprise a means which allows for recognition of the analyte. Such means can comprise a recognition component which is able to bind to the analyte. A particular example of a recognition component is a recognition molecule, such as a biorecognition molecule. Such molecules can be attached to the binding reagent in numerous ways, for example covalently or through passive absorption.

As used herein, the term “analyte” refers to any molecule, compound or particle the presence of which or amount of which is to be detected and wherein said molecule, compound or particle can bind to the binding reagent of the present invention. Suitable analytes include organic and inorganic molecules, including biomolecules. In a preferred embodiment, the analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, peptides, DNA and fragments thereof, nucleotides, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells); or spores. In a further preferred embodiment, the analyte is a cardiac marker such as brain natriuretic peptide (BNP), N-terminal related BNP, atrial natriuretic peptide, urotensin, urotensin related peptide, myoglobin, CK-MB, troponin I or troponin T.

In general, the binding reagent comprises a plurality of cleavable species which, when cleaved, are detectable using electrochemical means. There are therefore two characteristics which must be shown by the cleavable species. Firstly, they must be able to be cleaved from the binding reagent and, secondly, once they have been cleaved, they must be detectable using electrochemical means.

As used herein, the term “electrochemical means” refers to any method which involves oxidation and/or reduction at an electrode surface which can be used to determine the presence and/or amount of an electrochemically active species, also known as an electroactive species.

The cleavable species may show electrochemical activity when they have been cleaved from the binding reagent. Alternatively, the cleavable species may be transformable, once they have been cleaved from the binding reagents, into an electrochemically active species. As a further alternative, the cleavable species, after being cleaved from the binding reagent, can result in further species becoming electrochemically active. The presence of these further species can then be detected using electrochemical means and the presence and/or amount of the cleaved species thus determined. Preferably the cleavable species is not electrochemically active when attached to the binding reagent.

The provision of more than one cleavable species per binding reagent provides the possibility for amplification of the resulting signal. If there is, for example, an amplification of 10⁶ of the signal then a picomolar level of analyte may give rise to a signal which is equivalent to a micromolar level of analyte. Such amplification provides a convenient means by which to measure low levels of analyte. Typically, each binding reagent comprises at least 10⁴ cleavable species. Preferably, each binding reagent comprises at least 1 cleavable species. More preferably, each binding reagent comprises at least 10⁶ cleavable species.

The binding reagent may be labelled with an electroactive species or may be provided with a binding region to which the electroactive moiety may become attached.

The labelled binding reagent may be chosen such that the label is electrochemically active when cleaved from the binding reagent, or capable of being transformed into an electrochemically active species, or causing a further species to become electrochemically active. Preferably the labelled species is not electrochemically active when attached to the binding reagent.

The electroactive species may be any that is capable of being oxidised or reduced at an electrode surface. The electroactive species may be a redox reagent and therefore capable of being repeatably oxidised and reduced at an electrode surface. The binding reagent may be labelled with a plurality of electroactive moieties. Provision of more than one electroactive moiety per binding reagent provides the possibility for amplification of the resulting signal. Thus for example an amplification of 10 power 6, a picomolar level of analyte may give rise to a signal which is equivalent to a micromolar level of analyte. This provides a convenient means by which to measure low levels of analyte.

The cleavable species may comprise any moiety which can be detected using electrochemical means. Examples of such moieties include those derived from ferrocene, nitrophenol, aminophenol, hydroquinone, salicylic acid and sulphosalicylic acid. Further examples of such moieties are ferrocene aldehyde, ferrocene carboxylic acid, 4-nitrophenol, p-aminophenol, m-nitrophenol, hydroquinone, salicylic acid and sulfo-salicylic. Preferred moieties are those derived from ferrocene. Examples of such derivatives ferrocenes are those which carry groups derived from aldehyde, methylketone, ethylketone, hydroxymetlhane, hydroxyethane, methyl(hydroxy imine), carboxylic acid, carboxy phenyl carboxylic acid and carboxy propanoic acid. Preferably, the cleavable species are derived from ferrocene aldehyde.

Further examples of moieties which can be detected by electrochemical means which could present in the cleavable species are methylene blue, colloidal gold, naphthoquinone-4-sulphonate, p-N,N-dithylaminophenylisothiocyanate, p-aminophenylphosphate (PAPP), p-nitrophenylphosphate, 3-indoxyl phosphate (3-IP), N-(10,12-pentacosadiynoic)-acetylferrocene, silver on colloidal gold labels, hydroquinone diphosphate (HQDP), 4-amino-1-naphthylphosphate, 1,4-dihydroxy and 1,4-hydroxy-amine derivatives, p-aminophenyl beta-D-galactopyranoside, hydroquinone, 3,3′,5,5′-tetramethylbenzidine, cymantrene, TMB(Ox), 1-naphthyl phosphate, naphthol, indigo, ascorbic acid 2-phosphate (AAP) and 2,3-diaminophenazine.

The electrochemical moiety may be any that is suitable for the purposes of conducting an assay test. An example of such is ferrocene and derivatives thereof. The electrochemical species may have various substituents or modifications in order to make suitable for use, such to affect its solubility in the fluid sample of interest, to affect the redox potential, to reduce or eliminate binding to components that may be present in the fluid sample, to make it stable and so on.

Cleavage of the electrochemical species may be done in a number of different ways such as by exposure to light of a particular wavelength, by use of an enzyme, or chemically such as for example cleavage by use of an acid. The chemical cleavage reagent may itself be photogenerated. Typically, the cleavable species are photocleavable or acid cleavable. Of the above, cleavage by light is preferred as it does not require the addition of further reagents which may interfere with the assay. Light may be applied to a discrete region of the assay device, for example the capture zone. Furthermore, the direction and positioning of the light beam may also be easily controlled by the use of lenses, filters, baffles and so on.

One or more detection electrodes may be provided as part of the device and may be situated in close proximity to the capture electrode. Provision of the electrodes in close proximity allows for a large capture efficiency of the cleaved electrochemical species.

The binding reagent advantageously comprises a plurality of attached cleavable labile species. Accordingly, when the labelled binding is captured at a capture zone a large number of redox groups may be cleaved from the binding reagents thus providing amplification of the signal.

Suitable cleavable groups include disulfide bonds, ortho-nitrobenzenes, diols, diazo bonds, ester bonds, sulfone bonds, acetals, ketals, enol ethers, enol esters, enamines and imines.

In one embodiment of the invention, the labile group is a photolabile group, which may comprise an aromatic nitro group, and in particular an aromatic nitro group wherein the nitro group is in the ortho position. Thus, in one embodiment, the cleavable groups comprise an ortho-nitrobenzyl derivative.

Suitable acid cleavable groups include disulphide bonds, t-butyl esters of carboxylic acids and t-butyl carbonates of phenols.

Alternatively, the labile group may be an acid labile group that may be cleaved by the production of an acid from a photoacid generator. In one embodiment of the invention, an acid labile group may be treated with a photoacid generator prior to exposure to light.

In one embodiment, the cleavable species comprises a moiety which can be detected using electrochemical means as defined above and a cleavable group as defined above. An example of such a cleavable species is one which comprises a derivative of ferrocene aldehyde and an ortho-nitrobenzene derivative.

In order to provide a binding reagent with a large, for example greater than 10 power 6 of labile species, various means may be adopted. One such means is to provide a central core, such as a polymer particle as a site by which to attach binding reagents and/or labelled species. In this regard, in one embodiment, the present invention relates to a binding reagent which comprises a central core. This central core can act as an anchor point to which the cleavable species can be attached. This attachment can be either direct, i.e. the cleavable species are connected to the central core without the use of an intermediary, or indirectly, i.e. the cleavable species are connected to the central core via an intermediary. Suitable central cores include polymer spheres, such as those comprising latex, gold nanoparticles and hydrogels. A further example of a central core is a microcrystalline particle. A preferred central core is a latex bead. In order to attach the cleavable species to the central core, either directly or indirectly, the core can be modified. Suitable modification includes aldehyde-, carboxylic acid- and amino-modification. Aldehyde modification is preferred. In a preferred embodiment, the central core is an aldehyde-modified latex particle. Typically, the central core will be from 5 to 5000 nm, preferable from 10 to 1000 nm and more preferable from 50 to 500 nm.

Another way of achieving a high number of labile species is to attach them to a linear, branched or coiled polymer chain such as dendrimers, an interpenetrating polymer network (IPN). The polymer chain(s) may be attached to a base substrate such as a particle, forming a polymer brush, or other species in which the polymer chains extend from the substrate. One or more binding species may also be attached to the polymer chain(s) or substrate and so on. In one embodiment, the present invention relates to a binding reagent which comprises at least one dendritic or polymeric moiety. Typically, the cleavable species are attached to the dendritic or polymeric moiety. Suitable dendritic and polymeric moieties include such moieties to which the cleavable species can be attached. Examples of suitable dendrimers include poly(amidoamine) PAMAM dendrimers, poly(propylene imine) dendrimers and phenylacetylene dendrimers. Examples of suitable polymers include dextran, PAMAM, PEI, PEG, polyelectrolyte and streptavadin. A preferred polymeric moiety is dextran. Suitable types of dextran have molecular weights ranging from 10,000 to 2,000,000 Da, preferably molecular weights ranging from 100,000 to 500,000 Da.

In general, the dendritic and polymeric moieties carry functional groups which allow for attachment of the cleavable species. These functional groups can either be present on the dendritic and polymeric moiety itself or can be introduced thereto. Suitable functional groups include amine, carboxylic acid/carboxylate, NHS ester, hydroxyl, aldehyde, maleimide, epoxy, thiol groups. A preferred functional group is an amine group. With regard to the polymeric moieties, the functional groups can be present on the polymer chain or can be introduced via a crosslinker. A preferred polymeric moiety is amino-dextran. The dendritic or polymeric moieties may also be attached to a central core or particle.

In one embodiment, the binding reagent of the present invention comprises at least one dendritic or polymeric moiety which is attached to a central core. In this embodiment, the central core is preferably an aldehyde-modified latex bead and the dendritic or polymeric moiety is preferably amino dextran. The cleavable moieties can be attached to the dendritic or polymeric moiety. This is an example of the cleavable moieties being attached to the central core indirectly with the dendritic or polymeric moiety acting as an intermediary.

The binding reagents of the present invention could also be produced by a layer by layer self-assembly method which involves consecutive deposition of oppositely charged polyelectrolytes. As used herein, a polyelectrolyte is a polymer having ionically dissociable groups. Examples of polyanions which may be present in the polyelectrolyte are polyphosphate, polysulfate, polysulfonate, polyphosphonate, polyacrylate. Examples of polycations which may be present in the polyelectrolyte are polyallylamine, polyvinylamine, polyvinylpyridine, polyethyleneimine. In order to produce such a binding reagent, the cleavable species could firstly be attached to one or more polyelectrolytes. This could be achieved, for example using functional crosslinkers. The polyelectrolytes could then be alternatively assembled with oppositely charged polyelectrolytes onto a central core. Suitable central cores are as defined above. Suitable polyelectrolytes include poly(allylamine hydrochloride) and poly(styrenesulfonate). After the polyelectrolytes have been assembled, recognition components could then be added.

One problem which the inventors have shown, is the issue of non-specific binding between the labile species and the binding reagent. The larger the number of labile species per binding reagent that are provided, the greater this problem becomes. The inventors have shown that spatial and or physical separation of the binding reagent from the labile species serves to reduce or eliminate non-specific binding.

Accordingly, the present invention also provides a binding reagent in which the cleavable species have dendritic or polymeric moieties on their outer surface. In this context, the outer surface of the cleavable species is considered to be part of them which, when the binding reagent is in a fluid sample, is able to interact with the fluid sample i.e. the outer surface of the cleavable species groups is that part of the cleavable species which is at the exterior of the binding reagent. Any polymeric or dendritic moiety which can reduce or eliminate non-specific binding can be used in this regard. Typical examples are dextran, PEG, a polyelectrolyte and streptavidin. A preferred polymeric or dendritic moiety is dextran.

The surface of the binding reagent can also be blocked with polymers or dendritic moieties such as PEG to decrease the non-specific binding.

According to one embodiment, a particle is provided with one or more polymer chains such as a dextran to which are attached a number of cleavable species forming an inner core. Surrounding this core is provided a further outer core comprising one or more polymer chains such as dextran to which is/are attached the binding species. Separation of the binding species from the cleavable species in this way has been shown to reduce non-specific binding. Other embodiments could be envisaged which provide separation of the binding reagent from the labile species.

A further problem which has been shown to arise when using protein containing biological samples is one of binding of the labile electrochemically active group to the proteins. One of the usual disadvantages normally associated with using ferrocene as an electrochemical group in biological samples is that ferrocene binds to albumin and other biological proteins in blood, which negates the effect of the electrochemical signal produced at the electrodes. The present inventors have overcome this problem by providing cleavable electrochemical molecules (i.e cleavable species) that upon cleavage yield a ferrocene derivative incorporating a ferrocene group and further additional groups that prevent or substantially prevent binding of the ferrocene moiety to hydrophobic regions of the proteins. As shown in more detail in the examples, ferrocene derivative N-{2-[2-(2-Amino-ethoxy]-ethyl}-ferrocamide, was particularly advantageous in this respect and provided a signal that was commensurate with the concentration of the analyte in the sample. Thus, the present invention also provides for binding reagents which comprise cleavable species wherein said cleavable species are modified such that, when cleaved, they do not interact with the analyte or other moiety involved in the assay. Such modification can be achieved, for example, by pegylation. In general, when pegylated each cleavable moiety will comprise from 1 to 100 moieties derived from ethylene glycol, preferably from 1 to 25 moieties and more preferably from 1 to 10 moieties. When the cleavable species comprises a ferrocene derivative, it has been found that pegylation using a chain derived from two ethylene glycol moieties was found to be effective.

The cleavable species may be attached to the particles using conventional surface attachment chemistry known to those of skill in the art. However, the ferrocene moiety was attached to the particles by conjugation of a thiol group to a malemido function to produce a thioether linkage. The malemido group may be attached to the surface of the particles using, for example, aminodextran, or a dendrimer.

The binding reagents of the present invention may comprise additional components such as solubilising agents, for example linear or branched PEG or sugar derivatives, which can promote the solubility of the cleavable species, both before and after cleaving, which can enhance the effectiveness of an assay which employs the binding reagent of the present invention.

An example of a moiety which can be present in the binding reagents of the present invention is shown below:

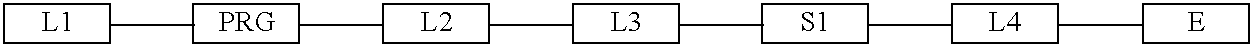

wherein:

L1 is a linker which comprises at least one functional group which can attach to a dendritic or polymeric moiety or the central core. Groups which can be present within L1 include amine, carboxylic acid/carboxylate, NHS ester, hydroxyl, aldehyde, maleimide, epoxy, thiol, halogen groups. The length of L1 can be controlled in order to improve the solubility of the cleavable species and/or the accessibility to the functional group(s);

PRG is a photoreactive group which can absorbed UV light in a wavelength range down to 340 nm. An example of such a group is a 2-nitrobenzyl derivative;

L2 is a linker which contains either a primary or secondary benzylic hydrogen. A secondary benzylic hydrogen is preferred for kinetic improvement of cleavage;

L3 promotes the cleavage at L2. Suitable groups for L3 include a carbamate, an ester, an amide linker;

S1 promotes the solubility of the photocleavable molecule and the cleaved derivative. Suitable solubilising moieties are linear or branched PEG, sugar derivatives;

L4 is a stable linker between the solubilising moiety S1 and the electrochemical group. Examples of suitable linkers are amide, ester, carbamate, ether, thioether groups; and

E is an electrochemical detectable group.

The above moiety is merely an example of one which may be present in the binding reagents of the present invention. Depending upon factors such as the mechanism of cleavage, the above example relates to photocleavage, the nature of the species when cleaved, in the above example the cleaved species is itself electrochemically active, and the requirements a particular assay, the actual moieties which are present in the binding reagent can altered accordingly.

The present invention provides for a binding reagent as defined herein. The present invention also provides for the use in an immunoassay of a such a binding reagent. The present invention further provides for an assay which comprises such a binding reagent.

In general, the binding reagents of the present invention are such that when bound to an analyte of interest they can be immobilized at a capture phase. Usually, such binding will not take place in the absence of the analyte. Immobilization at the capture phase can involve a second binding reagent which can itself be immobilized at the capture phase or, alternatively can be mobile. When mobile, the components will generally form a binding reagent-analyte-second binding reagent complex which can then be immobilized in the capture phase.

The assay may be a heterogeneous assay, such as a lateral flow or microfluidic type of assay wherein a binding reagent, analyte or analyte analogue is immobilised at the surface of a capture phase which serves to bind either directly or indirectly to a mobile labelled reagent. The labelled reagent (also referred to as the binding reagent) may be provided in the device prior to use or mixed with the fluid sample. The labelled reagent may also be one member of a binding pair such as an antigen or antibody.

The assay, device, kit and method of the invention rely on a capture phase that requires a binding reagent that is capable of binding to an analyte, and which binding reagent allows coupling of the labelled reagent. Once the labelled reagent has been immobilised at the capture phase, the electrochemical moieties or moiety may be cleaved from the reagent and detected at an electrode surface.

The capture phase may be provided for example on the surface of a particle, porous carrier or non-porous surface such as the inside surface of a microfluidic device. An example of a porous carrier is nitrocellulose or glass fibre. A particle may be for example a polymer sphere such as latex or a hydrogel. The non-porous surface could be chosen from any suitable material such as a plastic or glass and may be smooth or textured. The capture phase is suitably provided in a discrete zone, which may be referred to as a capture zone.

An assay device may have a capture zone in which is provided an immobilised binding reagent (also referred to at the second binding reagent) provided to which the mobile labelled binding reagent is capable of becoming either directly or indirectly attached. According to a further embodiment, both the unlabelled and labelled binding reagents (wherein the unlabelled binding reagent is also referred to as the second binding reagent) may be mobile and the device is provided with means by which to immobilise either the unlabelled binding reagent-labelled reagent complex or the unlabelled reagent-analyte-labelled reagent complex at a capture zone. The means maybe permit passage of the unbound labelled reagent but not the bound labelled reagent, for example a filter on the basis of size exclusion. The unlabelled binding reagent may for example be labelled with a particle having a size which does not allow it to pass through the filter whilst the labelled binding reagent is able to pass through said filter. Thus formation of an unlabelled binding reagent-labelled binding reagent complex immobilises the labelled binding reagent upstream from or at the filter. The size of the filter and particle may be chosen accordingly. The particle may for example be a hydrogel.

The device may be used in conjunction with a meter or may be an integral part of a meter. The device is typically intended to be disposable whilst a meter is intended to be reused. Where the meter and device are an integral unit, the meter may be disposable. The meter may contain one or more of the following: signal transduction elements, a light source, display means, signal processing means, means to receive or connect to the device, a power source, memory means and signal output and input means.

For the purposes of the invention, reference to a labelled binding reagent or to a labelled species attached to a binding reagent, does not necessarily imply that the binding reagent is attached directly to the label of interest. One or more labels and one more binding reagents may for example be attached to the same or a different further matrix such as a polymer or particle, thus effectively indirectly attaching or linking the labelled species and binding reagent. A binding reagent may comprise a binding species attached to a matrix.

The assay device and kit of the invention is suitable for the detection of a range of analytes in a fluid sample. The sample may be biological, environmental or industrial in nature. The biological sample may be derived from an animal or human. The sample may be any biological sample chosen from blood, serum, plasma, interstitial fluid, urine, cerebrospinal fluid, tears, saliva, nasal fluid and so on. The sample may a solid sample such as cellular debris, or cells which may be mixed with a liquid to provide a fluid sample.

One aspect of the invention provides for an assay device or kit for providing a measure of the amount or presence of an analyte in a sample, comprising;
(a) a binding reagent which is capable of binding to analyte of interest in the sample or to an immobilised reagent to form a binding pair,
wherein the binding reagent is labelled with a species having a labile group that is cleavable in response to a stimulus to provide a labile electrochemically active species,
(b) a capture phase comprising a support having a reagent which is capable of binding or attaching to said analyte or to said labelled reagent, and;
(c) an electrode capable of detecting the labile electrochemically active species to provide an indication of the presence or amount of analyte present.

The device may optionally be provided with additional reagents or means by which to cleave the labile species. Where the means of cleavage is for example by light, the device may be provided with a light source. Alternatively the light source may be provided in a meter, wherein the device is arranged to cooperate with the meter. The light source is positioned so as to illuminate the zone of interest, such as a capture phase or zone.

With respect to the use of light as the cleaving stimulus, the invention is particularly advantageous as the use of the kit only requires a single step to identify the concentration of the analyte, the application of light of a particular wavelength to cleave the labile bond, to provide an electrochemical measurement of the amount or presence of the analyte in the sample. The current provided from the oxidation and/or reduction of the electrochemical compound at the electrode surface may be correlated to the amount or presence of the analyte in the sample.

In one embodiment of the invention, the particles utilised in either the amplification or capture phases, or both, may be of any suitable particular substrate, such as latex, gold or silica beads. When the assay kit is utilised in conjunction with a microfluidic device, the particles of the amplification phase may, advantageously, be provided as a powder or as a printable ink, which may be provided on the surface of a microchannel, and which may be resuspended following passage of the sample therethrough.

The electrodes according to the invention may be constructed of any suitable material, such as palladium, platinum, gold, silver, carbon, titanium or copper. The electrodes are coated with an ion exchange membrane such as nafion, which is particularly advantageous when used in conjunction with, for example, ferrocene as the electrochemical redox group. The nation coating, advantageously, allows Fc⁺ ions to accumulate which may stripped from the electrode surface. The electrodes may be closely spaced, for example at a distance from 5 u from one another providing for the possibility of further amplification of the signal. The electrodes may be interdigitated.

The present invention is also concerned with labelled binding reagents for use in immuioassays as well as immunoassays, assay devices and kits thereof that can be utilised to identify or provide a measure of the amount of a desired analyte in a fluid sample. The present invention is also concerned with a meter which is designed to work in conjunction with an assay device and/or kit.

According to a first aspect, the present invention provides a labelled binding reagent for use in an immunoassay wherein the binding reagent is labelled with a labile species which may be cleaved from the binding reagent to produce a labile electrochemically active species which may subsequently be detected at an electrode surface.

According to a further aspect, the invention provides an immunoassay device for determining the presence or amount of an analyte in a sample wherein said device comprises a labelled binding reagent according to the previous aspect.

According to further aspect, the invention provides for an immunoassay kit comprising a reagent according to the first aspect.

According to yet a further aspect, the invention provides for a method of performing an immunoassay utilising a reagent according to the first aspect.

The present invention provides a binding reagent for use in an immunoassay wherein the binding reagent is labelled with one or more labile cleavable electrochemically active species attached to the binding reagent via a cleavable group. The present invention also provides such a binding reagent the cleavable group may be chosen from a photo cleavable group, and an acid cleavable group. The present invention further provides such a binding reagent wherein the electrochemically active species is a redox active species. This active species can be a ferrocene or ferrocene derivative. The present invention also provides such a binding reagent wherein the binding reagent is provided with a plurality of labile cleaveable electrochemically active groups.

The present invention also provides a method of detecting the presence or amount of analyte in a fluid sample, comprising mixing a fluid sample suspected of containing the analyte of interest with a binding reagent labelled with one or more labile electrochemically active groups and a second binding reagent to form a second binding reagent-labelled binding reagent complex which is immobilised in a capture zone, cleaving the one or more electrochemically active groups from the immobilised complex and subsequently detecting the electrochemically active groups at an electrode surface to provide an indication of the amount or extent of analyte or present in the fluid sample.

The present invention further provides an assay kit for providing a measure of the amount or presence of an analyte in a sample, comprising;
(a) a binding reagent which is capable of binding to analyte of interest in the sample or to an immobilised reagent to form a binding pair,
wherein the binding reagent is labelled with a species having a labile group that is cleavable in response to a stimulus to provide a labile electrochemically active species,
(b) a capture phase comprising a support having a reagent which is capable of binding or attaching to said analyte or to said labelled reagent, and;
(c) an electrode capable of detecting the labile electrochemically active species to provide an indication of the presence or amount of analyte present.

The present invention also provides such assay kit where an electrode is provided in the vicinity of the capture zone. Such an electrode can be coated with an ion-exchange membrane. An example of such an ion-exchange membrane is nation.

The following examples illustrate the invention.

### EXAMPLES

### 3.1 Design of the Amplification Vehicle/Phase

### 3.1.1 UV-Cleavable Electrochemical Molecule

o-Nitrobenzyl derivatives have been widely used in organic synthesis in particular as a protecting group and in biological applications for separating, purifying and identifying target biomolecules because of their high photocleavage efficiency by low energy UV-light.

A supposed photolysis mechanism of o-Nitrobenzyl derivatives is shown in scheme 3.1. It is suggested the aci-nitro intermediate A, which is in rapid equilibrium with a cyclic form B) is formed in a three steps procedure:
1) Activation of the nitro group by UV-light
2) iitramolecular hydrogen transfer from benzylic carbon to the oxygen in the nitro group.
3) Electron rearrangement.
   Then the released of compound D and the formation of the nitroso derivative C occurred by oxygen transfer from nitrogen to benzylic carbon.

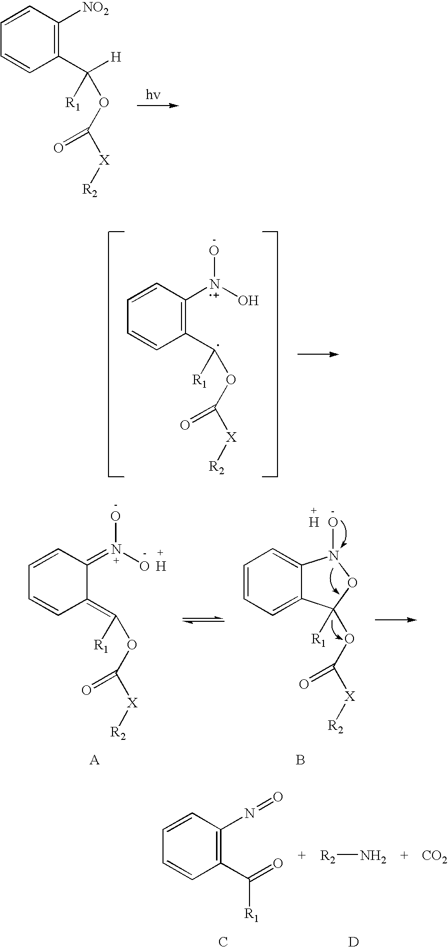

Scheme 3.1: Suggested mechanism of photolysis of o-Nitrobenzyl derivatives.

We decided to apply this photocleavage property as a tool for the design of an electrochemical assay where the electrochemical signal would be initiated by the UV-cleaving of a labile bond.

### 3.1.2 Synthesis of the UV-Cleavable Ferrocene Molecule

Our first aim was to synthesis a new molecule which contains an O-Nitrobenzyl core, a functional group allowing the attachment of this molecule onto a support, an electrochemical group and a photocleavable bond which could be cleaved with high efficiency under UV illumination in order to rapidly release an electrochemical derivative into solution.

One example of this molecule is represented below.

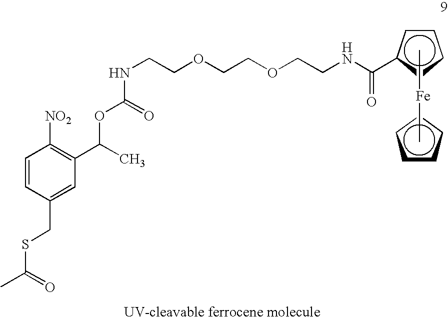

The synthesis of the UV-cleavable ferrocene molecule 9 is shown in scheme 3.2. The precursor 1-(5-Bromomethyl-2-nitro-phenyl)-ethanone 4 was obtained in 5 steps starting from the commercially available 5-Methyl-2-Nitrobenzoic acid according to Doppler et al. methodology. The ketone 4 was then converted to its corresponding secondary alcohol 5 on treatment with sodium borohydride. Subsequently, the thiol group was introduced as its thioacetate form, which served as a protecting group during the introduction of the ferrocene derivative 8.

This ferrocene derivative 8 was obtained according to scheme 3.3 by direct coupling of ferrocene carboxylic acid to a large excess of 2,2′-(Ethylenedioxy)bis-(Ethylamine). The excess was used in order to favour the formation of the monoalkylated product at the expense of the disubstituted one. Afterwards, the primary amino function of 8 was coupled to the reactive (N-hydroxysuccimide) ester to form a carbamate bond.

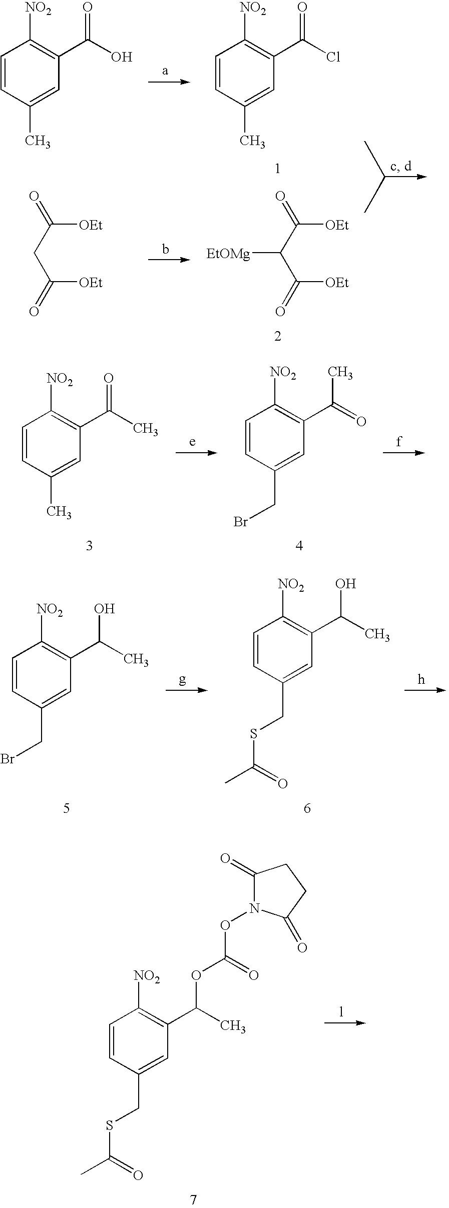

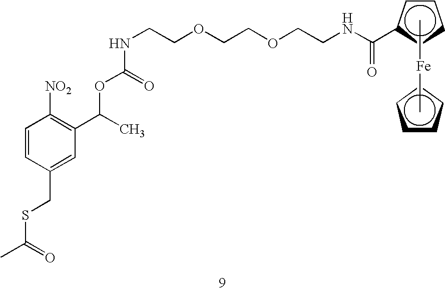

Scheme 3.2: Reagents and conditions: a) SOCl₂, CH₂Cl₂; b) Mg, EtOH, toluene, reflux; c) Toluene, reflux; d) H₃O⁺, reflux; e) NBS, Benzoylperoxide, CCl₄, reflux; i) NaBH₄, dioxane/methanol; g) CH₃C(O)S K⁺, DMF; h) DSC, Et₃N, CH₃CN; i) 8, Et₃N, CH₂Cl₂

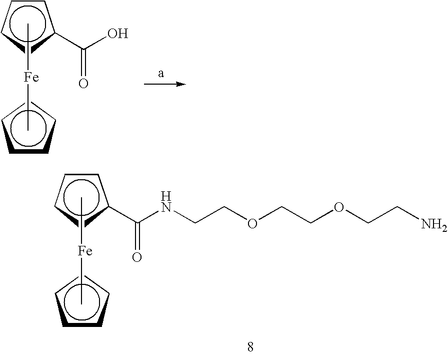

Scheme 3.3: Reagents and conditions: a) EDCI, HOBt, ET₃N, H₂N(CH₂CH₂O)₂CH₂CH₂NH₂, CH₂Cl₂.

### 3.1.3 Photolysis in Solution

As the molecule was synthesised, our next objective was to demonstrate its photocleavage in solution.

According to scheme 3.1 (section 3.1.1), the photolysis (hv: 365 rin) of the UV-cleavable ferrocene 9 should result in the formation of two main products (scheme 3.4). The ferrocene derivative 8 can be either protonated or not according to the pH of the middle.

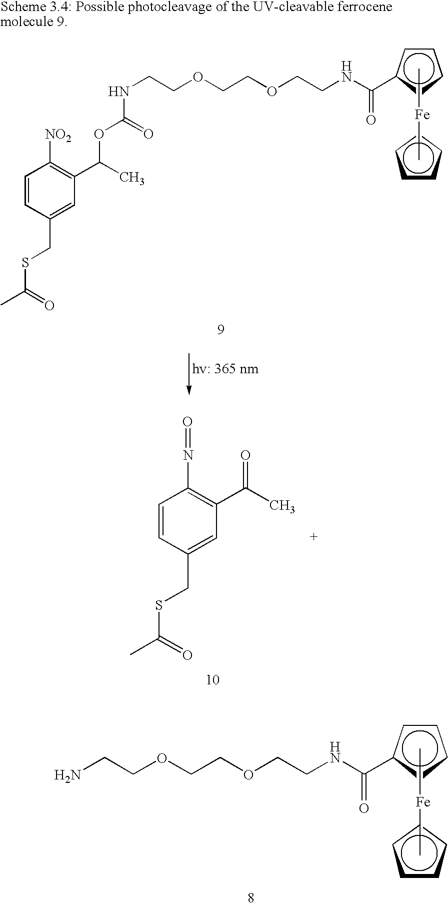

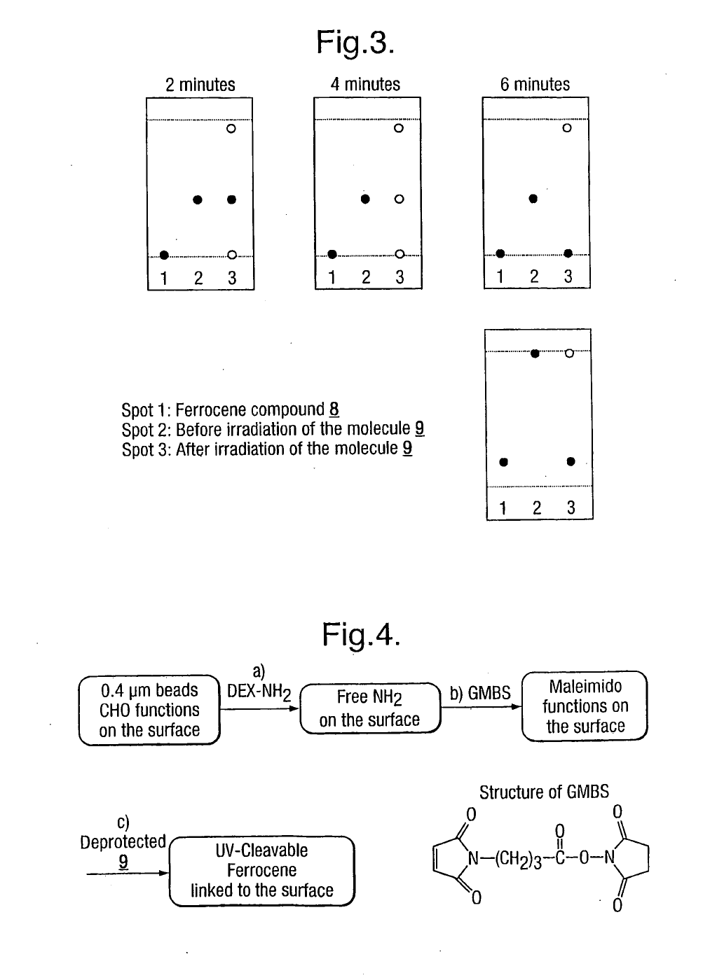

The cleavage study was followed by Thin Layer Chromatography (TLC) after irradiation for a definite time of a methanolic/PBS solution of the UV-cleavable ferrocene 9 (FIG. 3).
After 2 minutes of irradiation, the appearance of two new products was observed; One of them corresponding to the ferrocene product 8 (on the base line), the other one corresponding probably to the nitroso-derivative 10 just under the front line).
Under the irradiation conditions used, the Uv-cleavable ferrocene molecule was completely cleaved in less than 6 minutes.

### 3.2 Attachment of the UV-Cleavable Ferrocene Molecule to a Support/Photocleavage from the Support

Our next objective was to evaluate the efficiency of the cleavage of the molecule 9 while attached to a support. As seen in the introduction part, many of different supports could be considered as much as they contain a large number of attachment sites. In our example we choose 0.4 μm latex particles which are aldehyde modified.

### 3.2.1 Attachment of the UV-Cleavable Ferrocene Molecule to the Latex Particles

With the purpose of detecting analytes dove to pM scale, a large number of UV-cleavable ferrocene needed to be attached per particle. Therefore, surface modifications were considered in order to increase the number of available attachment sites onto the particles.

The actual UV-Cleavable Ferrocene Molecule 9 has a protected thiol, which after deprotection presents a reactivity that allows its conjugation to a maleimido function leading to a thioether linkage (scheme 3.5).

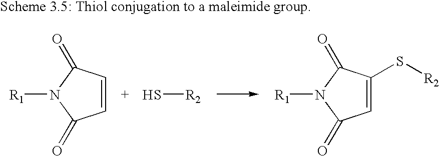

Therefore, we decided to explore the ways of introducing several maleimide groups at the surface of the particles.

One example of the surface modification used is shown in FIG. 4.

The attachment of the UV-cleavable ferrocene was achieved in 3 steps starting from the commercially available 0.4 μm beads (1.6% solids, Polymer Microspheres, Red fluorescent, Aldehyde modified). In a first step, Amino-Dextran was coupled to the beads by reductive amination. Because of the polymeric nature of the Amino-Dextran it was expected that remaining uncoupled amino functions would still be available at the surface of the latex for further coupling. Thus, in a second step maleimide groups were introduced using the heterobifunctional cross-linker GMBS (maleimidobutyryloxy-Succinimide ester). Finally, after deprotection of the thiol according to scheme 3.6, the UV-cleavable ferrocene molecule was covalently coupled to the latex via thioether linkages.

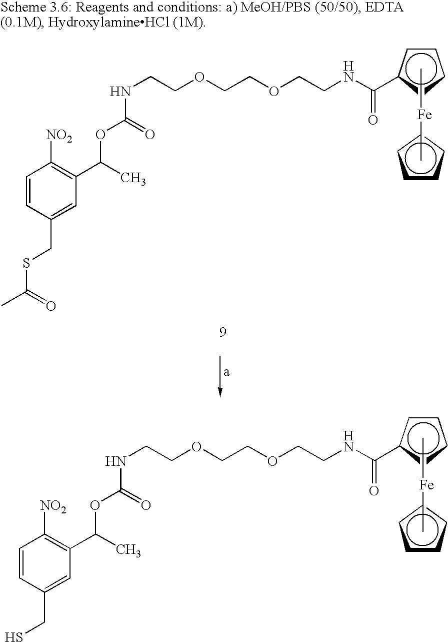

3.2.2 Photocleavage of the UV-Cleavable Ferrocene Molecule from the Latex Particles

### 3.2.2.1 Photocleavage Using a UV Lamp Model B100A

The photocleavage from the beads and therefore the released in solution of the ferrrocene derivative 8 was then studied using cyclic voltammetry. The irradiation was performed for 5 minutes on different bead concentrations using a UV lamp model B100A with a wavelength of 365 nm (intensity of 8,900 μW/cm² at 10″).

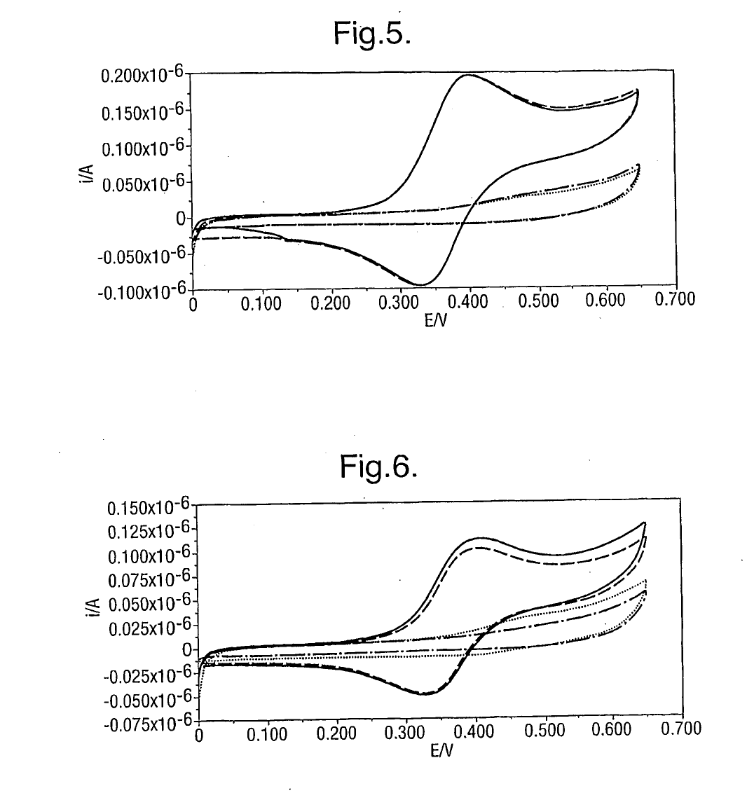

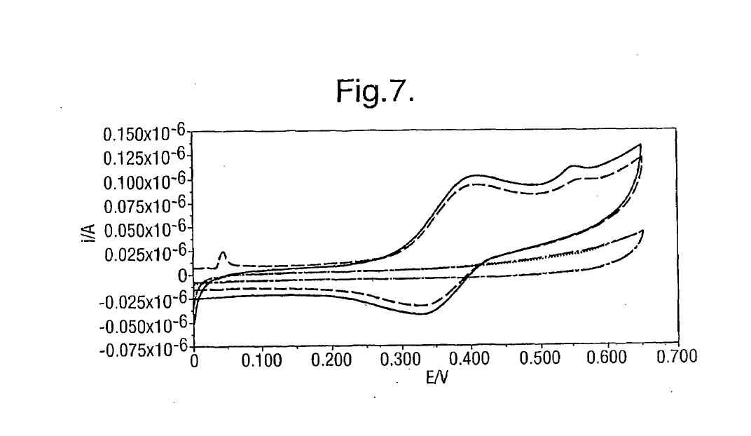

The difference in the electrochemical response before and after irradiation shown in FIGS. 5, 6 and 7, was attributed to the photoreleased from the latex particles of the ferrocene derivative 8.

### 3.3 Investigation of Properties of 400 nm TRL Beads Sensitised with UV Cleavable Ferrocene Molecule

Experiments were focused on following the cleavage of the UV cleavable ferrocene molecule real time (instead of a single point measurement e.g. cyclic voltammetry to allow greater understanding of the cleavage process) and to determine an approximate value for the number of UV cleavable ferrocene molecules per bead.

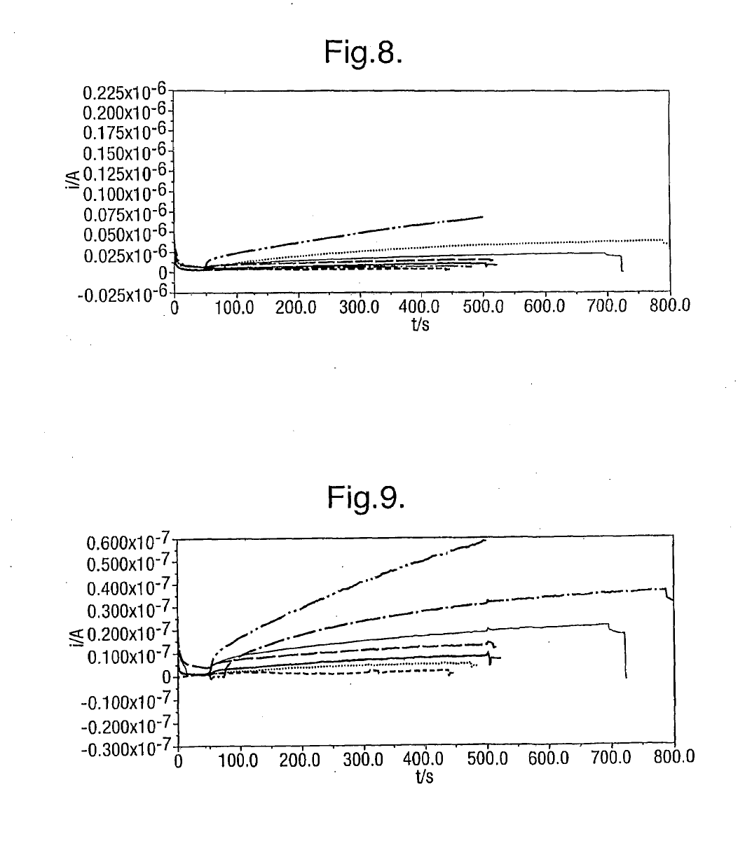

A number of bead solutions were prepared whose concentrations were subsequently determined by flow cytometry measurements (1.16E+08, 46600000, 23300000, 11650000, 5825000, 2912500 and 1456250 beads per 17 μL). The experimental details are described in section 2.5.2. The results are summarised in FIG. 8, the raw data is shown including the PBS control. Approximately 50 seconds into the chronoamperometry measurements the green LED was turned on providing UV light at 360 nm. Interestingly an initial increase in current is observed even if no UV cleavable ferrocene molecule is present (i.e. PBS control), at present the origin of this phenomenon is not known. The current measured is logical with respect to bead concentration and hence the amount of ferrocene molecule cleaved, with the highest bead concentrations resulting in the highest current and the lowest bead concentrations resulting in the lowest current.

The same data after normalisation (subtraction of the PBS data to baseline correct) and resealing is shown in FIG. 9. The current dependency on bead concentration is more clearly shown. It must be noted that the current is still increasing when the chronoamperometry measurements are terminated in the case of four highest bead concentrations (1.16E+08, 46600000, 23300000, 11650000) indicating incomplete UV cleavage within the given time scale.

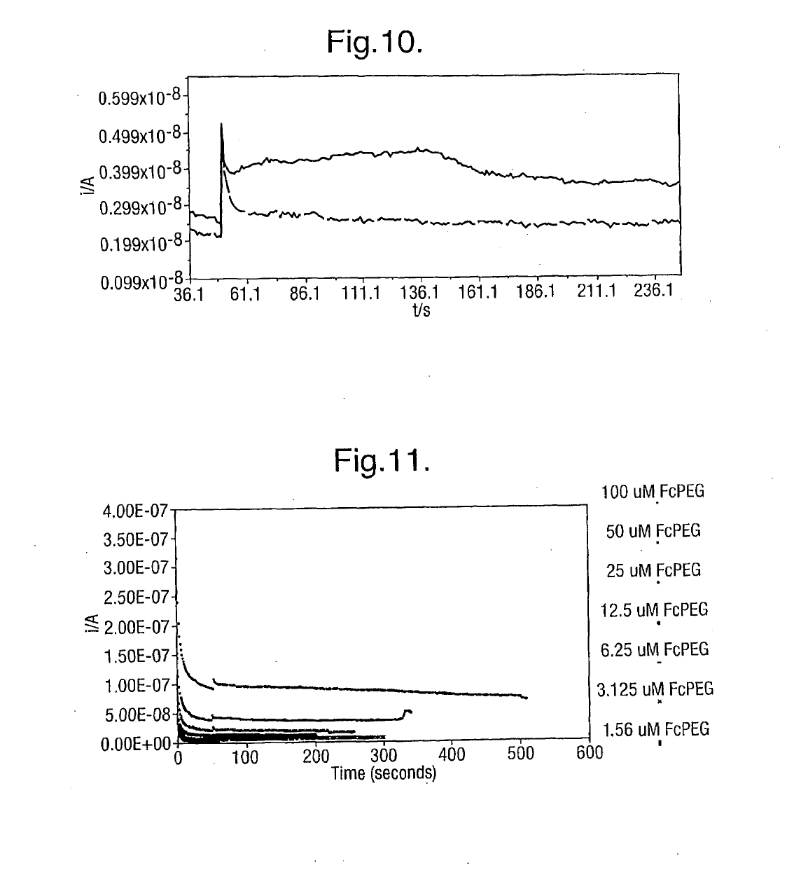

The lowest concentration of beads (2912500 beads per 17 μL) shows an increase in current followed by a decrease in current indicating that the UV cleavable ferrocene molecule is becoming depleted as shown in FIG. 10, in comparison the PBS control shows no such behaviour.

In order to calculate approximately how many UV-cleavable ferrocene molecules there was per 400 nm bead a calibration curve of current vs. UV cleaved ferrocene molecule 8 was performed. This involved measurement of the current response over a range of known concentrations of the UV cleaved ferrocene molecules using identical methodology use to investigate bead concentration and current magnitude. The data is summarised in FIG. 11.

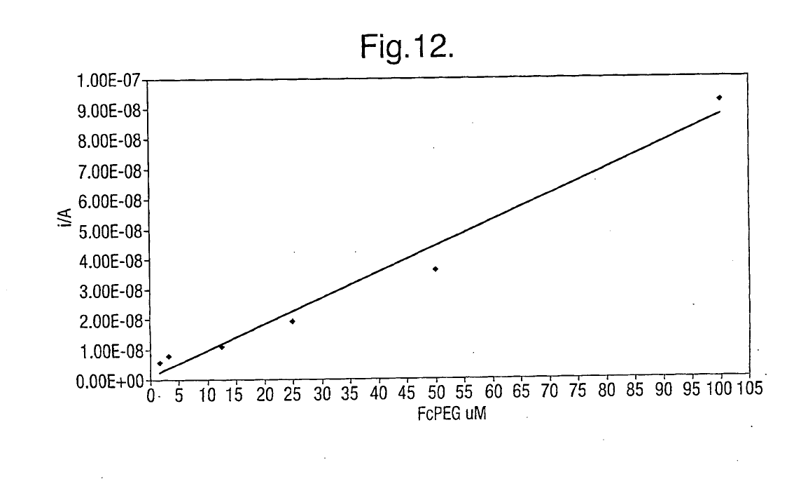

From FIG. 11 a calibration curve of current vs. concentration of the UV cleaved ferrocene molecule could be derived. Values (i/A) were extracted from the 200 second points of FIG. 9 for each concentration. The resultant calibration curve is shown in FIG. 12.

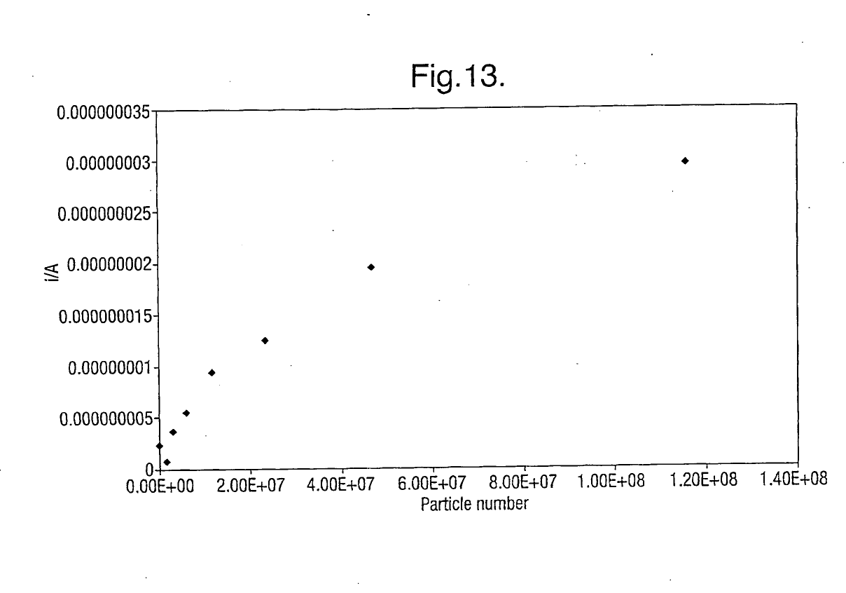

A plot of particle number vs. i/A (UV cleaved ferrocene molecules) clearly shows a good relationship between the two parameters. This is shown in FIG. 13.

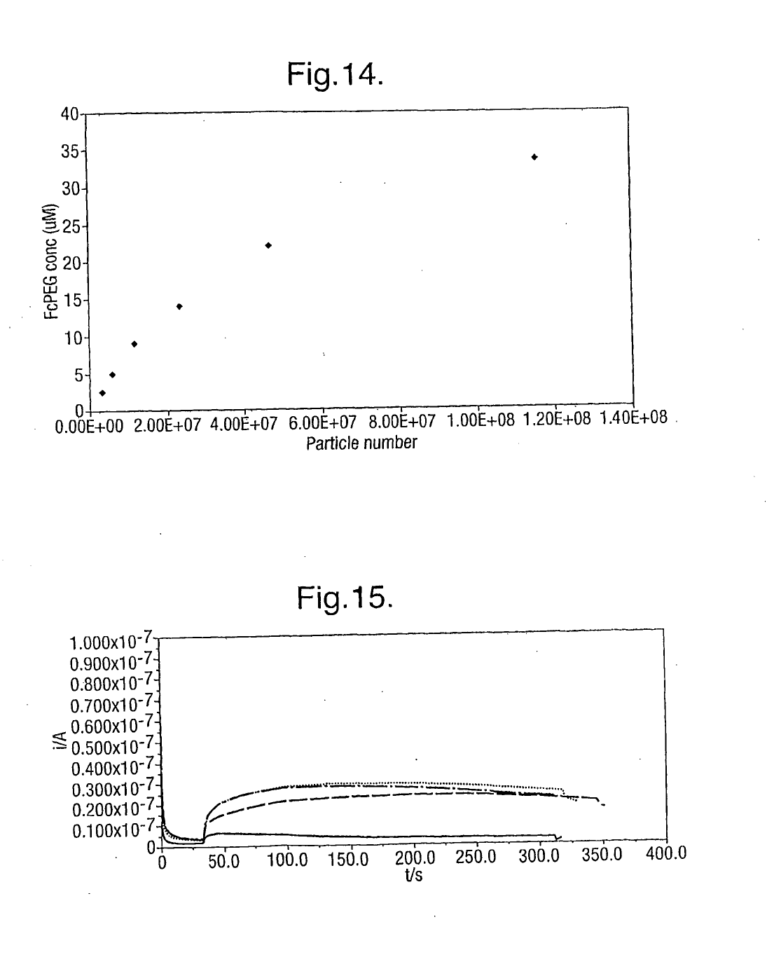

The calibration curve (FIG. 12) allowed the conversion of the current from the UV cleaved ferrocene molecules from the 400 nm beads in FIG. 10 to concentration so a plot of UV cleaved ferrocene (μM) vs. bead concentration can be obtained as shown in FIG. 14.

Using FIG. 14 an approximate value of number of UV-cleavable ferrocene molecules per 400 nm bead can be calculated. The calculation is shown below:

Use of 22 μM value from FIG. 14.

Number of particles per ml=2.74E+09 per ml−as determined by flow cytometry

Number of particles per μL=2.74E+06 per μL

Number of particles per 17 μL=1.16E+08

Number of molecules per 1 μM=6.02E+17

Therefore number of molecules per ml=6.02E+14

Therefore number of molecules per μL=6.02E+1

Therefore number of molecules per 17 μL=1.02E+14

Therefore in 17 μL@22 μM=2.25E+14 FcPEG molecules

$$Approximate number of FcPEG molecules per particle = 2.25 E + 14 / 1.16 E + 08 = 4.83 E + 06 per particle$$

This approximate number is an underestimation as the current was still increasing at the 200 second point and it must also be noted that only the UV cleavable ferrocene molecule was coupled to the 400 nm beads. Once antibody is also coupled to the 400 nm bead the number of molecules of UV cleavable ferrocene molecules will significantly decrease.

### 3.4 UV Cleavage of Ferrocene Molecules in Thin Layer Cells/Capillary Fill Devices

UV cleavage of ferrocene molecules from 400 nm beads was demonstrated above, however these measurements were made by applying drops of solution to screen printed electrodes with a total volume of 17 μL. In section 3.4 we demonstrate very similar measurements using thin layer cells/capillary fill devices.

As described in the materials and methods section a double sided adhesive tape and a cover slip was used to construct a thin layer cell/capillary fill device upon a screen printed electrode. A summary of the results is shown in FIG. 15.

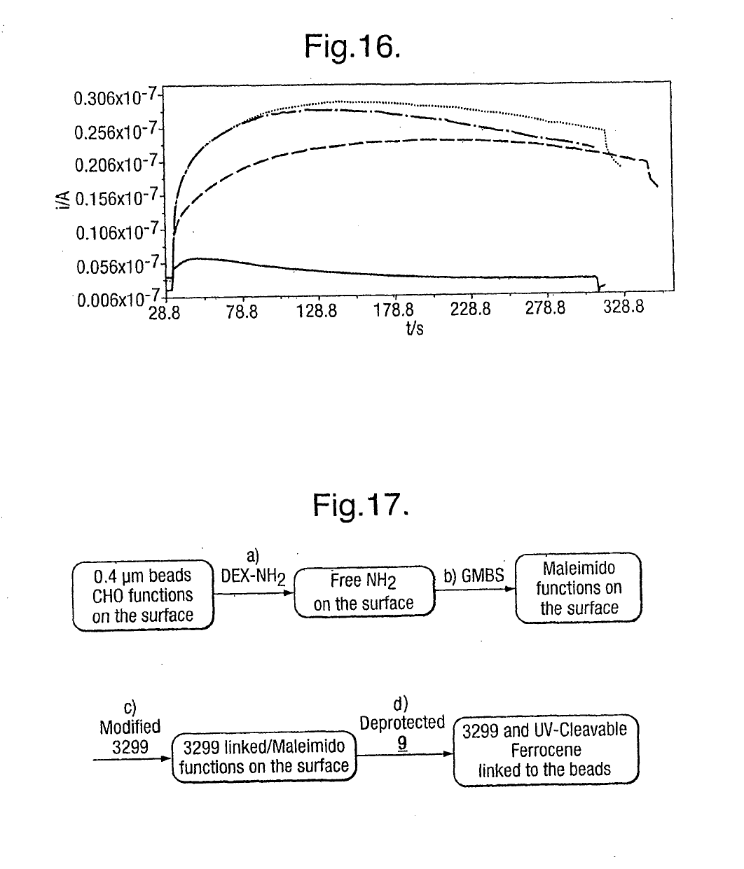

The same data is shown in FIG. 16 but resealed. The simple experiment demonstrates the UV cleavage can be performed in thin layer cells/capillary fill device with low sample volumes, although a 6 μL sample volume was used approximately only 2 μL covers the electrodes. In addition by changing the LED input voltage and hence the amount of UV light the amount of cleavage can be changed.

### 3.5 Coupling of Both the UV-Cleavable Ferrocene Molecule and the Antibody to the Latex Particle

Two opposing strategies were explored in order to couple both the UV-cleavable molecule and the antibody (3299 in this example):
First approach: coupling of the antibody to the support followed by the attachment of the UV-cleavable ferrocene molecule.
Second approach: attachment of the UV-cleavable ferrocene molecule followed by the coupling of the antibody.

### 3.5.1 Approach 1: Coupling of the Antibody Followed by the UV-Cleavable Molecule

Once again, surface modifications needed to be considered in order to couple a maximum of antibodies and UV-cleavable molecules to the beads.

One example of the surface modification used in this approach is shown in FIG. 17.

The coupling of both the antibody and the UV-cleavable ferrocene molecule was achieved in 4 steps using the same chemistry as described above to attach the UV cleavable ferrocene to the latex particles. Steps 1, 2 and the deprotection of the UV-cleavable molecule 9 were explained also in this section. In a third step, the 3299 antibody, which was modified according to scheme 3.7, was conjugated to the maleimide groups linked to the beads. Because of the large number of maleimido functions present at the surface of the particle and because of the bulky size of the antibody, it was expected that remaining maleimide groups would still be available for the coupling, in a fourth step, of the UV-cleavable ferrocene molecule.

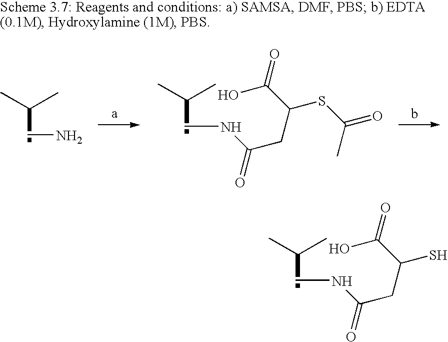

### 3.5.2 Approach 2: Coupling of the UV-Cleavable Molecule Followed by the Antibody

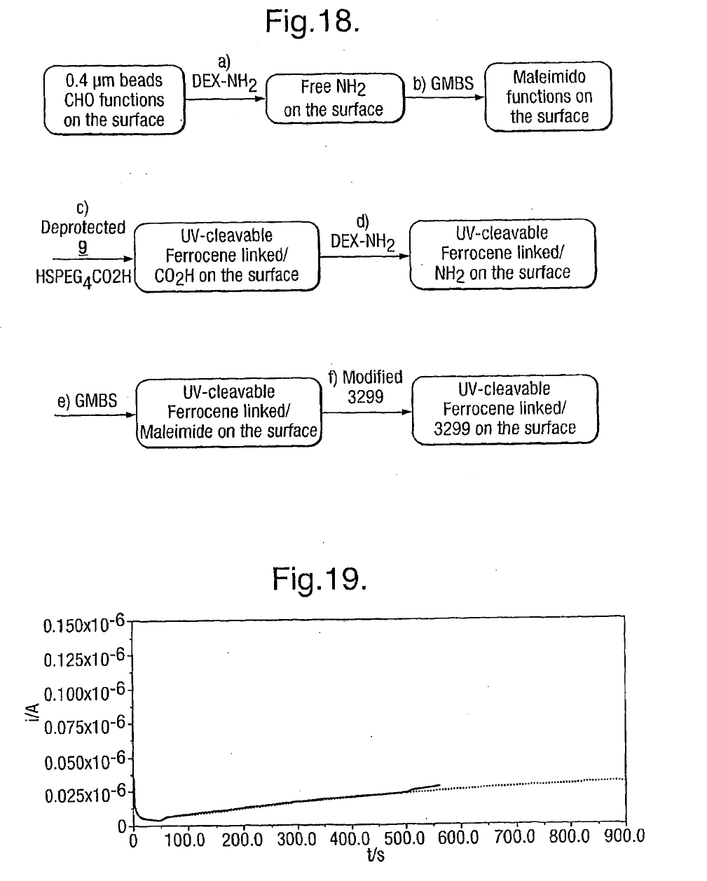

One example of the surface modification used in this approach is shown in FIG. 18. The coupling of both the V-cleavable ferrocene molecule and the 3299 antibody was achieved in 6 steps using a chemistry related to the one used in sections describing the attachment of the UV cleavable ferrocene to the latex particle (A) and in Approach 1 above. Steps 1, 2 and the deprotection of the UV-cleavable ferrocene 2 were explained in the section mentioned A above. The antibody modification was explained in Approach 1.

The strategy used here consisted of the attachment, at the same time, of both the UV-cleavable ferrocene molecule and a second bi functional linker in order to introduce available carboxylic functions at the surface of the latex for further coupling of antibodies. HSPEG₄CO₂H was chosen on this purpose. At this stage, a second layer of Amino-Dextran was coupled to the latex via an amide bond, followed by the attachment of the cross-linker GMBS whereby the modified 3299 was conjugated.

### 3.6 Investigation of Number of UV-Cleavable Ferrocene Molecules Per 400 nm Bead when Antibody is Also Coupled

An identical measurement and procedure as to that described in section 3.3 was used to determine the number of UV-cleavable ferrocene molecules when antibody is also coupled to the 400 nm bead. In particular two approaches were examined, firstly the 3299 (anti hCG) antibody was coupled first followed by coupling the UV cleavable ferrocene molecule and secondly the reverse scenario whereby the UV cleavable ferrocene molecule is coupled first followed by the antibody.

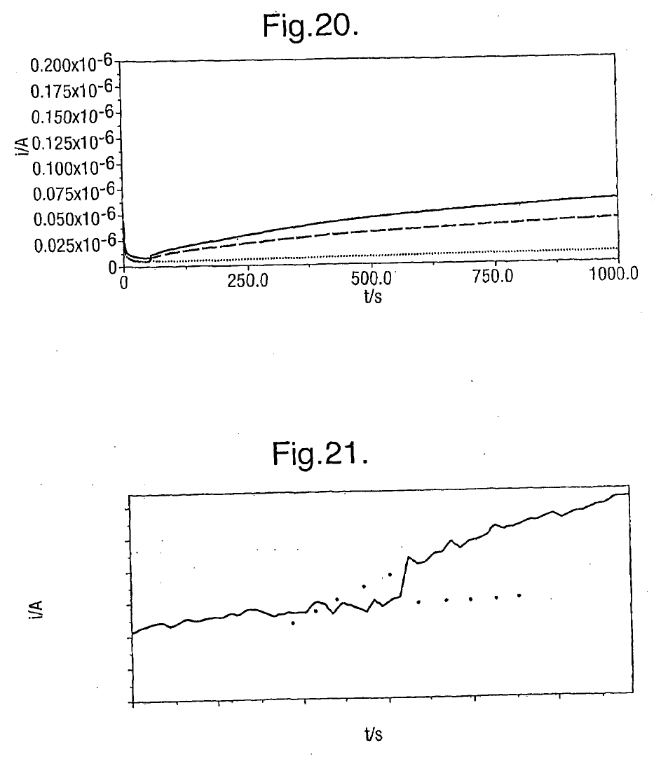

The results are summarised in FIG. 19 and FIG. 20.

The i/A values at 200 seconds were then used to calculate the number of UV cleavable ferrocene molecules per bead. The results are summarised in table 3.1

TABLE 3.1

| | Number of UV cleavable ferrocene |
|---|---|
| Experimental conditions | molecules per 400 nm TRL bead |
| Antibody coupled first followed | 3.78E+05 UV cleavable ferrocene |
| by UV cleavable ferrocene molecules | molecules |
| UV cleavable ferrocene molecules | 1.12E+06 UV cleavable ferrocene |
| coupled first followed by antibody | molecules |

Therefore coupling the UV cleavable ferrocene molecule to the 400 nm TRL bead first followed by the antibody yields the highest number of UV cleavable ferrocene molecules per 400 nm bead.

Interestingly during chronoamperometry measurement shown in FIG. 19 the LED input voltage was switched from 22 mV to 38 mV at approximately 504 seconds into the measurement. A change in rate is clearly observed as expected as emphasised in FIG. 21, confirming previous observations (see FIG. 15).

### 3.7 Design of the Capture Phase/Zone

As seen in the introduction the capture phase/zone must contain at least 2 well defined components: A surface and a biorecognition part which could either be passively absorbed to the surface or covalently attached after surface modifications.

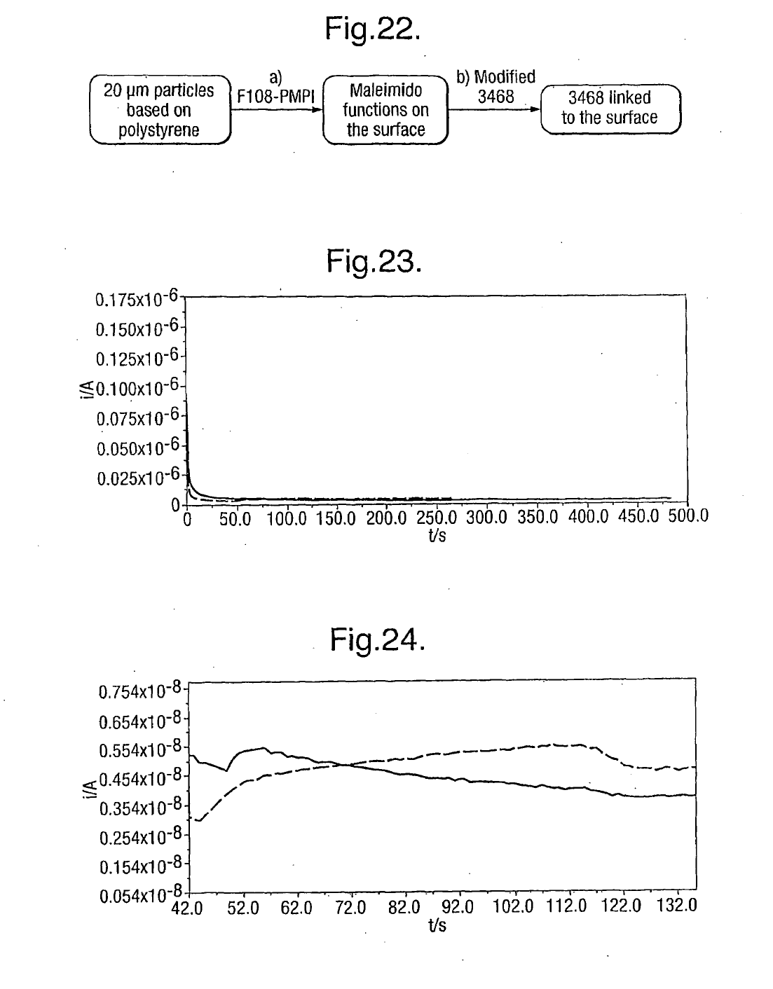

One example of a prepared capture phase is shown in FIG. 22.

In this example we choose to covalently attach the 3468 antibody to the modified 20 μm beads using a thioether linkage.

The coupling of the antibody was achieved in 2 steps starting from the commercially available 20 μm particles, based on polystyrene. In a first step, maleimide groups were introduced by absorption onto the surface of the beads of F108-IPMPI (for the synthesis see scheme 3.8 below), which is a triblock polymer detergent. The antibody 3468, modified according to scheme 3.7 section 3.5.1, was then conjugated to the maleimido functions.

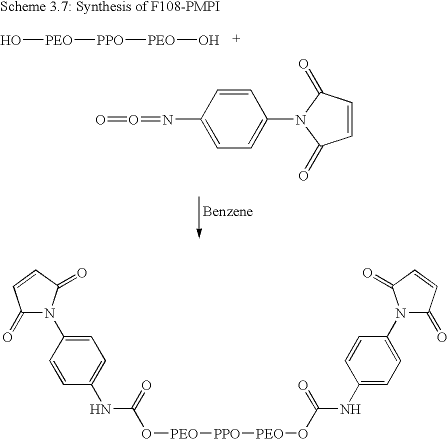

### 3.8 Chronoamperometry Measurements of hCG “Wet Assay” with IMF3

A wet assay was performed whereby the 20 μm particle, 400 nm particle and hCG standard (0 or 400 mIU) were premixed for approximately 30 minutes (see materials and methods for greater detail). Chronoamperometry measurements (see FIGS. 23 and 24) were performed using the IMF3 device (see materials and methods). Only one measurement of each concentration was performed due to the limited supply of 400 mm particles (anti-hcg antibody, UV cleavable ferrocene molecule) and ultimately UV-cleavable ferrocene molecule. Future studies will be reported when such particles become available. However, there is clearly a marked difference between the 0 and 400 mIUl hCG standards which is more clearly shown in FIG. 24. An initial increase in current is observed with the 0 hCG when the UV source is switched on followed by a subsequent decrease in current. In comparison the same initial increase is observed followed by a further increase in current which starts to decrease at approximately 117 second point. It is suggested that the solution is being depleted of UV cleaved ferrocene molecules.

### 3.9 Choice of UV Cleaved Ferrocene Molecule

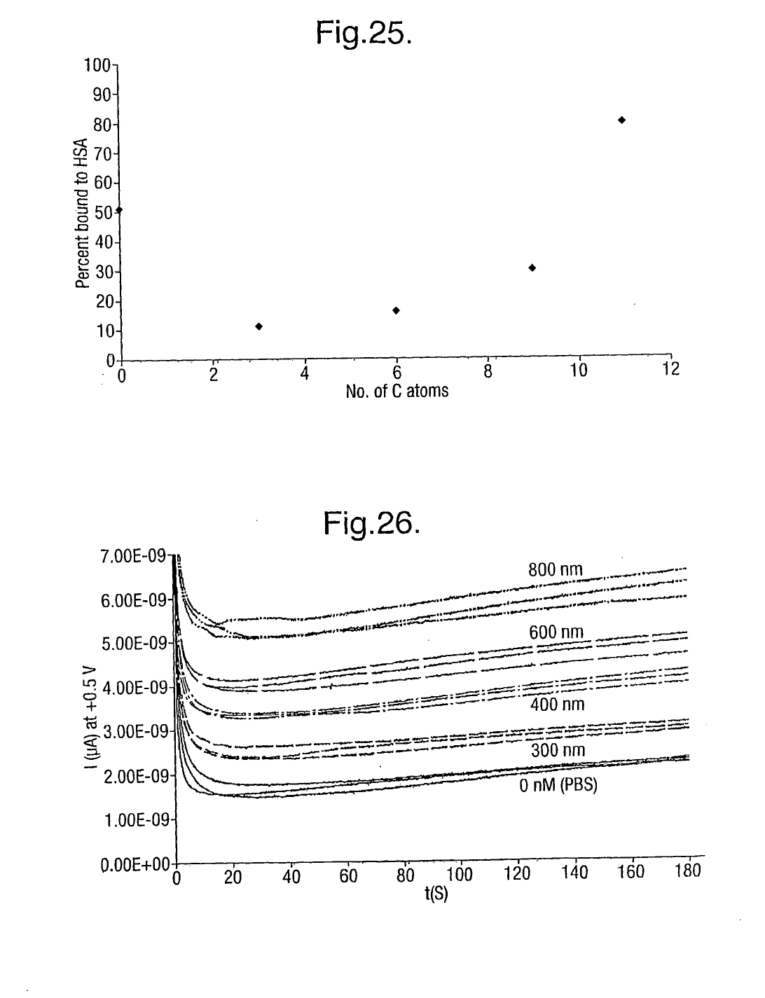

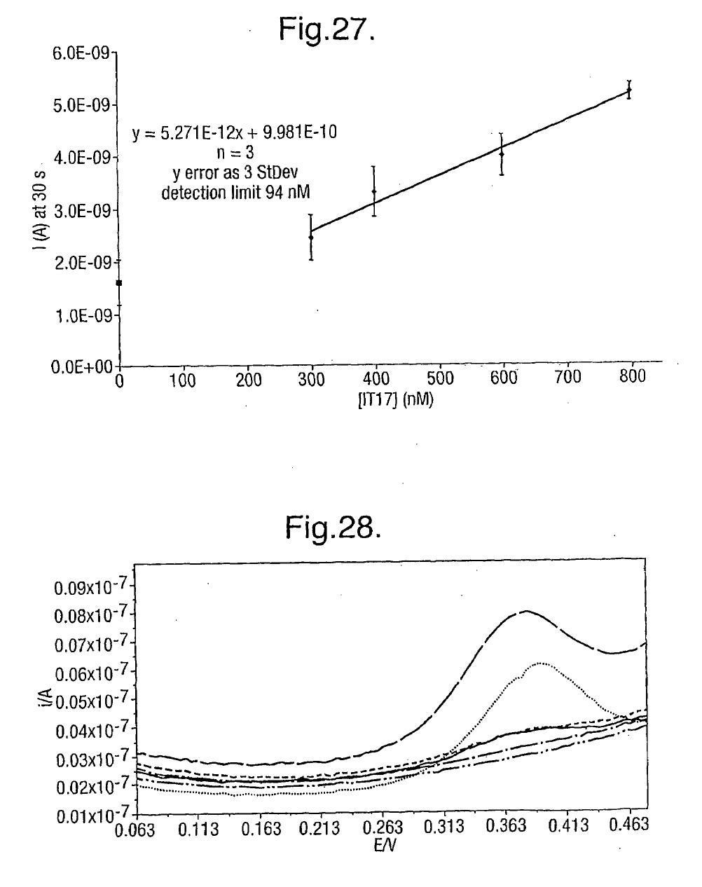

There are several different types of ferrocene molecules that could have been chosen for electrochemical measurement. Ferrocene PEG was the preferred molecule as previous experiments identified characteristics favourable for electrochemical measurement in protein, plasma or blood solutions. One of the problems of measuring electrochemical labels in such samples is the binding of electrochemical labels to protein molecules especially human serum albumin (HSA) and hence the loss of signal. A previous study investigating binding of ferrocene molecules to HSA is summarised in FIG. 25.

A series of ferrocene labelled fatty acid probes were synthesised that comprised of ferrocene, a linker, a solubilising spacer, a second linker and a fatty acid which differed in carbon length. The variation in the carbon length included 3 (compound 4), 6 (compound 6), 9 (compound), 11 (compound 2) and 16 (compound 10) carbon atoms including the terminal carboxyl group (scheme 3.8). Cyclic voltammetry was used to measure the concentration of the ferrocene labelled fatty acid probe with and without the presence of HSA allowing percentage bound to be calculated. FIG. 3.24 clearly demonstrates the percentage bound of the ferrocene labelled fatty acid probe species (25 μM) to HSA (500 μM) can be methodically controlled by varying the length of the carbon chain. Interestingly the zero carbon control molecule, ferrocene methanol is found to bind to HSA relatively strongly with 50% bound to HSA. Presently we are unsure where ferrocene binds to HSA and we have made no attempt to do so although it is suggested one of the drug binding sites may be involved and this is the subject of ongoing work. When the ferrocene is conjugated to a short carbon chain via a PEG linker molecule, this chain will prevent the ferrocene from binding to HSA, which may be due to steric hindrance or to a change in the charge on the ferrocene or a combination of both.

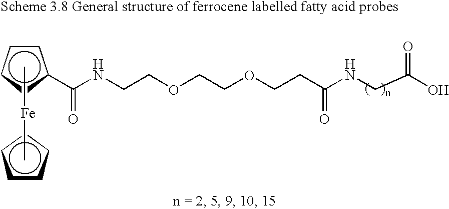

### 3.10 Electrochemical Measurement of UV Cleaved Ferrocene Molecules

Currently no attempt has been made to optimise the electrochemical measurement technique of the UV cleaved ferrocene molecules. Chronoamperometry was used throughout the study because it is a relatively simple measurement but also provides quality data e.g. kinetic data essential in the early development of potential electrochemical assays. There are however many other electrochemical measurements methodologies that allow for more sensitive measurement of ferrocene molecules. Previous studies have identified a number of electrochemical techniques than can be used to increase the measurement sensitivity. For example high sensitivity measurements of ferrocene molecules have been made with interdigitated miroelectrode arrays. Summary results are shown in figures with a sensitivity of 300 nM (sensitivity of measurement technique, not any assay linked to it).

Similarly differential pulse measurements have also been investigated as a possible measurement methodology of ferrocene molecules. The results are summarised in FIG. 28.

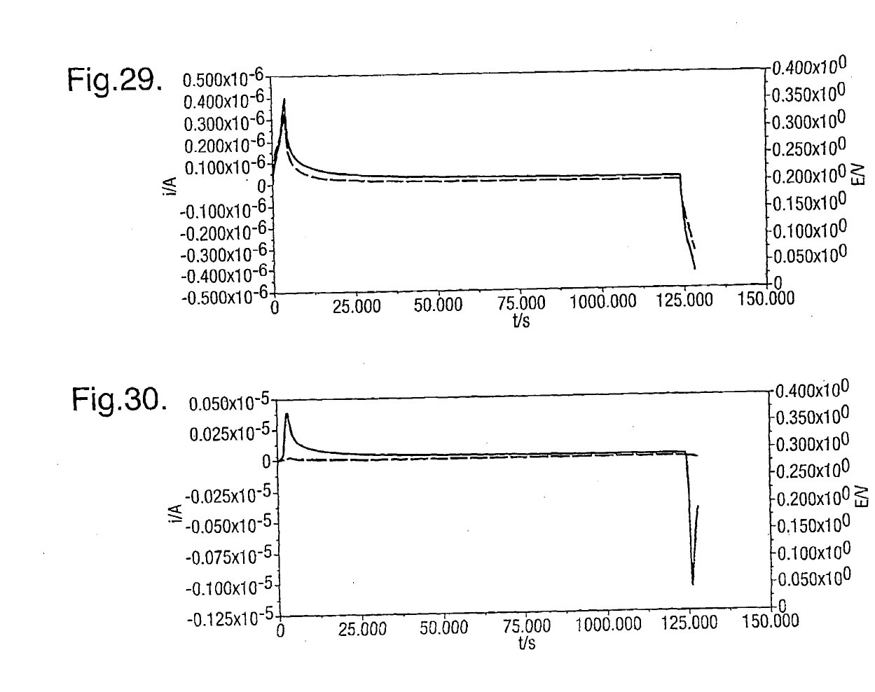

In addition, previous studies have shown that ferrocene molecules can be accumulated in nafion coated electrodes. In particular the reverse peak current was larger when the electrode was coated with nation than when the electrode was uncoated. This can be attributed to accumulation of the signal molecule in nation. To verify this, stripping voltammetry was carried out, where the potential was swept from 0V to a potential where the signal molecule is oxidised, subsequently kept there for two minutes and then swept back. From the current of the back scan it can be concluded that the signal molecule accumulates significantly in the nafion coating casted from water. No accumulation could be observed in the nafion which was casted from ethanol (see FIGS. 29 and 30).

In addition to the nafion membrane (cast from water) having ferrocene accumulation properties, it has also been shown to allow measurement of ferrocene compounds in the presence of uric and ascorbic acid. These compounds are two of the major electrochemical interferents found in blood. The nafion membrane allows the uric/ascorbic acid current contribution to be additive to the measured ferrocene current rather than “mediation” events occurring whereby the measured ferrocene current in the presence of uric/ascorbic acid is greater than the sum of the ferrocene and uric/ascorbic acid measured separately. The currents are however still additive and a background measurement of uric/ascorbic acid current contribution would need to be performed to background correct.

### Materials and Methods

All moisture-sensitive reactions were performed under a nitrogen atmosphere using oven-dried glassware and dried solvents. Unless otherwise indicated, reagents were obtained from commercial suppliers and were used without further purification. Reactions were monitored by TLC on Kieselgel 60 F₂₅₄ plates with detection by WV. Flash column chromatography was carried out using silica gel 60.

¹H NMR spectra were recorded at 300 MHz or 400 MHz on a Bruker AMX-300 or AVANCE 400. ¹³C NMR spectra were recorded at 75 MHz or 100 MHz. Relative integral, multiplicity (s: singulet, d: doublet, t: triplet, m: multiplet) and coupling constants, in Hz, were assigned where possible.

Mass spectra were obtained using a Micromass Quattro LC instrument (ES).

Reactions from step 6 were performed in the dark. The final product and all the intermediates were kept in the dark.

### 2.1 Synthesis of the UV-Cleavable Ferrocene Molecule

### Step 1: Synthesis of 5-Methyl-2-nitro-benzoyl chloride 1

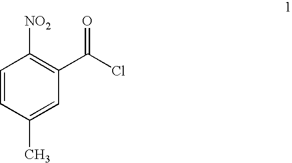

To a solution of 5-Methyl-2-Nitrobenzoic acid (1.50 g, 8.28*10⁻³ mol) in 20 ml of dry dichloromethane was added two drops of dry DWM and thionyl chloride (1.82 ml, 2.48*10⁻² mol). The solution was stirred at room temperature for 30 min. The solvent was then evaporated and the residue dissolved in 20 ml of ether, the solvent was then removed. The crude intermediate 1 was used without purification.

### Step 2: Synthesis of Ethoxymagnesium Diethyl malonate

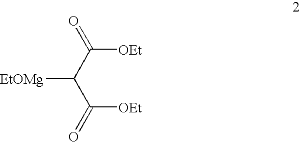

A reaction mixture was prepared consisting of Magnesium turning (0.294 g, 1.21*10⁻² mol), Diethyl malonate (1.84 ml, 1.21*10⁻² mol), ethanol (1.21*10⁻² mol) in 10 ml of dry toluene. The mixture was heated to reflux for 1 h30. Most of the magnesium was consumed over this period of time. This material was used directly. Comments: Used of a drying tube. If the reaction has not begun after 10 min (self-sustained vigorous reflux), 4 drops of carbon tetrachloride were added to the mixture.

### Steps 3 and 4: Synthesis of 1-(5-Methyl-2-nitro-phenyl)-ethanone 3

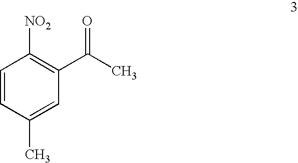

Intermediate 1 was dissolved in 5 ml of dry toluene and added to the solution of intermediate 2. The reaction mixture was refluxed for 30 minutes. The solvent was then evaporated and 45 ml of 6M sulphuric acid was then added to the residue. The mixture was refluxed for 3 h. After cooling down, the mixture was poured into a separatory flinnel and extracted with diethyl ether. After a basic washed, the organic phase was washed with water then dried over sodium sulfate, filtered and the solvent removed under reduced pressure to afford 1.365 g (92%, over the four steps) of the product as an orange oil. This material can be used directly in the next step.

¹H NMR (CDCl₃, 300 MHz) δ_{H} 2.39 (s, 3H, CH₃), 2.45 (s, 3H, CH₃), 7.12 (1H, m, ArH), 7.29-7.32 (m, 1H, ArR), 7.98-8.02 (m, 1H ArR).

¹³C NMR (CDCl₃, 75.56 MHz) δ_{c} 21.2 (CH₃), 30.0 (CH₃), 124.2, 127.5, 130.8, 137.9, 143.0, 146.0 (Ar), 200.4 (CO).

### Step 5: Synthesis of 1-(5-Bromomethyl-2-nitro-phenyl)-ethanone 4

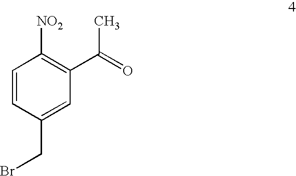

A mixture of 1-(−5-methyl-2-nitrophenyl)ethanone 3 (0.700 g, 3.91*10⁻³ mol), N-Bromosuccinimide (0.765 g, 4.30*10⁻³ mol) and benzoyl peroxide (10 mg) in 4 ml of dry carbon tetrachloride was heated to reflux for 1 h30. The mixture was then cooled down, filtered and evaporated to dryness.

Product purified by column chromatography (gradient Hexane/Ethyl acetate, 100% hexane to 80% hexane) to give 0.603 g (60%) of the title compound.

¹H NMR (CDCl₃, 300 MHz) δ_{H} 2.53 (s, 3H, CH₃), 4.48 (s, CH₂Br), 7.41 (m, 1H, ArH), 7.60 (m, 1H, ArH), 8.04 (m, 1H, ArH).

¹³C NMR (CDCl₃, 75.56 MHz) δ_{C} 30.0 (CH₃), 37.3 (CH₂Br), 125.0, 127.8, 131.0, 138.3, 144.8, 147.6 (Ar), 199.4 (CO).

m/z (+ES): 279.9 [M+Na]⁺.

### Step 6: Synthesis of 1-(5-Bromomethyl-2-nitro-phenyl)-ethanol 5

[symbol] Reactions carried out in the dark in order to avoid any contact with UV.

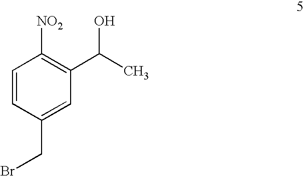

Solid Sodium Borohydride (0.081 g, 2.13*10⁻³ mol) was rapidly added to an ice cold solution of 1-(5-Bromomethyl-2-nitro-phenyl)-ethanone 4 (0.500 g, 1.94*10⁻³ mol) in dioxane (4 ml) and methanol (6 ml). After stirring 30 minutes at 0° C.; the remaining Sodium Borohydride was quenched by addition of acetone. The solvent was then evaporated. The crude was taken up into dichloromethane, washed with HCl/water and finally with brine. The organic phase was then dried over sodium sulfate, filtered and evaporated to dryness.

Product purified by column chromatography (1) Hexane/Ethyl acetate 90/10, 2) Hexane/Ethyl acetate 75/25) to give 0.353 (70%) of the title compound.

¹H NMR (CDCl₃, 300 MHz) δ_{H} 1.54 (m, 3H, CH₃CH), 4.49 (s, CH₂Br), 5.43 (m, 1H, CHCH₃), 7.45 (m, 1H, Arm), 7.85-7.88 (m, 2H, ArH).

¹³C NMR (CDCl₃, 75.56 MHz) δ_{C} 24.4 (CH₃CH), 38.5 (CH₂Br), 66.5 (CH₃CH), 125.1, 128.1, 128.6, 141.8, 143.7 (Ar).

m/z (+ES): 281.9 [M+Na]⁺.

### Step 7: Synthesis of Thioacetic acid S—[3-(1-hydroxy-ethyl)-4-nitro-benzyl]ester 6

[symbol] Reaction carried out in the dark in order to avoid any contact with UV.

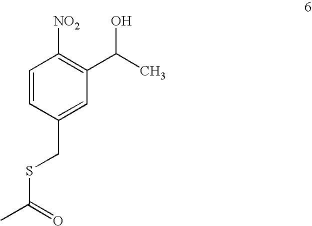

To a solution of 1-(5-Bromomethyl-2-nitrophenyl)-ethanol 5 (0.350 g, 1.35*10⁻³ mol) in 5 ml of dry DMF was added potassium thioacetate (0.170 g, 1.49*10⁻³ mol). The mixture was stirred at room temperature for 2 hours. The solution was then partitioned between water and dichloromethane. The organic layer was then washed with brine, dried over sodium sulfate, filtered and evaporated to dryness.

Product purified by column chromatography (gradient Hexane/Ethyl acetate, 100% hexane to 70% hexane) to give 0.242 g (70%) of the title compound.

¹H NMR (CDCl₃, 300 MHz) δ_{H} 1.55 (d, J=6.3 Hz, 3H, CH₃CH), 2.36 (s, 3H, CH₃CO), 4.14 (s, 2H, CH₂), 5.41 (m, 1H, CH₃CH), 7.31-7.34 (m, 1H, ArH), 7.75 (m, 1H, ArH), 7.83-7.86 (m, 1H, Arh).

¹³C NMR (CDCl₃, 75.56 MHz) δ_{C} 24.2 (CH₃CH), 30.3 and 32.8 (CH₂S+CH₃CO), 65.6 (CH₃CH), 124.9, 127.9, 128.4, 141.5, 144.4 (Ar), 194.5 (SCO).

m/z (+ES): 278 [M+Na]⁺.

### Step 8: Synthesis of Thioacetic acid 3-[1-(2,5-dioxo-pyrrolidin-1-yloxycarbonyloxy)-ethyl]-4-nitro-benzyl ester 7

[symbol] Reaction carried out in the dark in order to avoid any contact with UV.

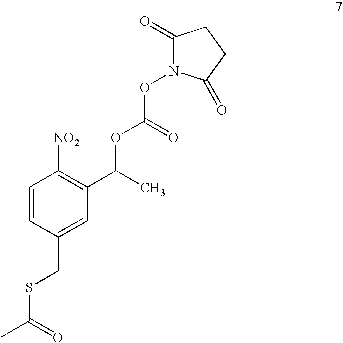

To a solution of Thioacetic acid S—[3-(1-hydroxy-ethyl)-4-nitro-benzyl]ester 6 (0.220 g, 8.66*10⁻⁴ mol) in 3 ml of dry acetonitrile was added triethylamine(2 eq). Then N′,N′-Disuccinimidyl carbonate (0.288 g, 1.126*10⁻³ mol) was added. The mixture was stirred at 0° C. for 30 min and then at room temperature overnight. The solvent was then evaporated under reduced pressure.

Product purified by column chromatography (gradient Hexane/Ethyl acetate, 100% hexane to 40% hexane) to give 0.171 g (50%) of the title compound.

¹H NMR (CDCl₃, 400 MHz) δ_{H} 1.75 (d, J=6.4 Hz, 3H, CH₃CH), 2.32 (s, 3H, CH₃CO), 2.77 (s, 4H, CH₂ succinimidyl), 4.16 (s, 2H, CH₂S), 6.38 (m, 1H, CH₃CH), 7.40, 7.61, 7.94 (m, ArH).

¹³C NMR(CDCl₃, 100 MHz) δ_{C} 21.9 (CH₃CH), 25.4 (CH₂ succinimidyl), 30.1 and 32.5 (CH₂S+CH₃CO), 75.8 (CH₃CH), 125.2, 127.1, 129.4, 136.1, 145.4, 150.5 (Ar), 165.5, 169.0, 194.2 (CO).

m/z (+ES): 419.1 [M4+Na]⁺.

### Step 9: Synthesis of Thioacetic acid S—[3-(1-{2-[2-(2-ferrocenoylamino-ethoxy)-ethoxy]-ethylcarbamoyloxy}-ethyl)-4-nitro-benzyl]ester 2

[symbol] Reaction carried out in the dark in order to avoid any contact with UV.

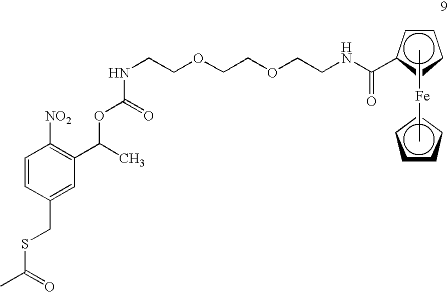

Thioacetic acid 3-[1-(2,5-dioxo-pyrrolidin-1-yloxycarbonyloxy)-ethyl]-4-nitro-benzyl ester 7 (0.150 g, 3.8010⁻⁴ mol) was dissolved in dry dichloromethane and added to a stirred solution of 8 (0.164 g, 4.56*10⁻⁴ mol) in dry dichloromethane. Triethylamine (1.2 eq) was then added. The mixture was stirred at room temperature overnight. The organic layer was washed with brine, dried over sodium sulfate, filtered and evaporated to dryness.

Product purified by column chromatography: Eluent:1) Ethyl acetate/Hexane (30/70), 2) Ethyl acetate/Hexane (60140), 3) 100% Ethyl acetate to give 0.097 g (40%) of the title compound.

[symbol]

The product was stored in the dark at 4° C.

¹H NMR (CDCl₃, 400 MHz) δ_{H} 1.59 (d, J=6.5 Hz, 3H, CH₃CH), 2.35 (s, 3H, CH₃CO), 3.33 (m, 2H, CH₂NH), 3.50-3.60 (m, 10H, CH₂O+CH₂NH), 4.11 (s, 2H, CH₂S), 4.20 (m, 5H, Cp), 4.33 (m, 2H, Cp), 4.67 (m, 2H, Cp), 5.30 (br, 1H, NH), 6.22 (m, 2H, CH₃CH+NH), 7.31 (m, 1H, ArH), 7.52 (m, 1H, ArH), 7.85 (m, 1H, ArH).

¹³C NMR (CDCl₃, 100 MHz) δ_{C} 22.1 (CH₃CH), 30.2 and 32.7 (CH₂S+CH₃CO), 39.2 and 40.7 (CH₂NH), 68.1-75.9 (several signals, Cp+CH₂O+CHCH₃), 124.9, 127.4, 128.4, 139.0, 144.1, 146.8 (Ar), 155.2 (CO carbamate), 170.3 (COCp), 194.1 (SCO). m/z (+ES): 664.2 [M+Na]⁺.

### Step 10: Synthesis of N-{2-[2-(2-Amino-ethoxy)-ethoxy]-ethyl}-ferocamide 8

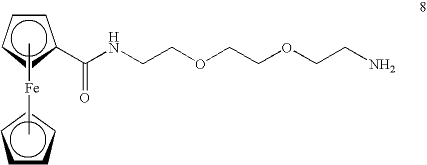

To a solution of ferrocene carboxylic acid (0.500 g, 2.17*10⁻³ mol) in dry dichloromethane was added 1-Hydroxybenzotriazole hydrate (0.326 g, 2.87*10⁻³ mol). After 10 min of stirring at room temperature EDCI (0.457 g, 2.87*10⁻³ mol) and triethylamine (2.2 eq) were added. The mixture was stirred at room temperature for 30 min. This solution was then added dropwise to a solution of 2,2′-(Ethylenedioxy)bis-(Ethylamine) (3.21 g, 2.17*10⁻² mol) at 0° C. The mixture was stirred at room temperature for overnight. After filtration, the filtrate was washed three times, dried over sodium sulfate, filtered and evaporated to dryness.

Product purified by column chromatography (eluent: dichloromethane/methanol/triethylamine, 85/10/5) to give 0.312 g (40%) of the title compound.

¹H NMR (CDCl₃, 300 MHz) δ_{H} 2.87 (m, 2H, CH₂NH₂), 3.48-3.61 (m, 10H, CH₂O+CH₂NH), 4.17 (m, 5H, Cp), 4.29 (m, 2H, Cp), 4.69 (m, 2H, Cp), 6.38 (br, 1H, NH).

¹³C NMR (CDCl₃, 75.56 MHz) δ_{C} 39.0 (CH₂NH₂), 41.2 (CH₂NH), 68.0-75.8 (several signals, Cp+CH₂O), 170.1 (CO).

m/z (+ES): 383 [M+Na]⁺.

### 2.2 Coupling of UV-Cleavable Ferrocene Molecule to Particles.

2.2.1 Coating of 400 nm TRL Beads (CHO Functions) with Aminodextran/Theoretical Latex Concentration 0.3% Solids

To a suspension of 400 nm TRL beads (CHO function, 187.5 μl, 1.6% w/v) in 392.5 μl of MES (pH 6.0, 50 mM) was added 400 μl of a solution of aminodextran obtained by dissolving 3 mg of aminodextran into 600 μl of N4ES (pH 6.0, 50 mM). The suspension was agitated using a bench vortex, 20 μl of a solution of NaBH₃CN (1 M) was then added. The latex was incubated overnight at room temperature with stirring (end-over-end mixer). The suspension was then spun (15,500 rpm, 15° C.) for 20 min. The supernatant was discarded, 1 ml of MES (pH 6.0, 50 mM) was added, the pellet was re-suspended using a bench vortex and an ultrasonic bath. The suspension was spun (15,500 rpm, 15° C.) for 20 minutes. The supernatant was discarded. This washing step was repeated 2 more times. Finally, the pellet was resuspended in 1 ml of MES (pH 6.0, 50 mM), sonicated and stored at 4° C. The final concentration of the aminodextran coated latex was in theory 0.3% (w/v).

### 2.2.2 Attachment of the UV-Cleavable Ferrocene Molecule

[symbol] Reaction carried out in the dark in order to avoid any contact with UV.

### 2.2.2.1 Introduction of the Crosslinker (Maleimido Butyryloxy-Succinimide Ester):

The suspension of aminodextran-latex (1 ml, prepared according to section 2.2.1) was spun (15,500 rpm, 15° C.) for 20 min. The supernatant was discarded, the pellet was re-suspended in 900 μl of PBS (pH 7.0) using a bench vortex and an ultrasonic bath. 5 mg of the GMBS crosslinker in solution in 100 μl of DMF was added to the latex and the suspension was incubated for 45 min at room temperature with stirring (end-over-end mixer). The suspension was then spun (15,500 rpm, 15° C., 20 min). The supernatant was discarded, the pellet re-suspended in 1 ml of PBS (pH 7.0) using a bench vortex and an ultrasonic bath. The suspension was spun (15,500 rpm, 15° C., 20 min).

The pellet was re-suspended in 325 μl of PBS (pH 7.0). 175 μl of DMF was then added (agitation) followed by 500 μl of a solution of the deprotected UV-cleavable ferrocene molecule (the deprotection of the UV-cleavable ferrocene molecule 9 was performed as described below in section 2.2.2.2). After sonication, the latex was incubated overnight at room temperature with stirring (end-over-end mixer). The suspension was then spun (15,500 rpm, 15° C., 20 min). The supernatant was discarded, 1 ml of a solution of 35% DMF in PBS was added, the pellet was re-suspended using a bench vortex and an ultrasonic bath. After agitation for 30 min at room temperature, the suspension was spun (15,500 rpm, 15° C., 20 min). The supernatant was discarded. This washing step was repeated 2 more times, The pellet was then re-suspended in a solution of 20% DMF in PBS, sonicated. After agitation for 20 min at room temperature, the suspension was spun (15,500 rpm, 15° C., 20 min). The supernatant was discarded. This washing step was repeated 1 more time. The pellet was then re-suspended in 1 ml of PBS, sonicated and the suspension was spun (15,500 rpm, 15° C., 20 min). The supernatant was discarded. Finally the pellet was re-suspended in 1 ml of PBS, sonicated and stored in the dark at 4° C.

### 2.2.2.2 Deprotection of the UV-Cleavable Ferrocene Molecule 9

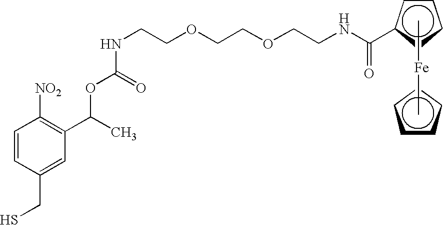

The UV-cleavable ferrocene molecule 2 (3 mg, 4.68*10⁻⁶ mol) was solubilized in 500 μl of methanol. 400 μl of PBS, 40 μl of EDTA (0.1 M) and finally 80 μl of hydroxylamine.HCl (1 M) were added. The mixture was stirred for 30 min at room temperature. Dichloromethane (4 ml) was then added. The mixture was poured into a separatory funnel, the organic phase collected and the solvent removed under reduced pressure. The deprotected UV-cleavable ferrocene molecule was then solubilized in 200 μl of DMF. 300 μl of PBS (pH 7.0) was then added (if the solution became cloudy few more drops of DMP could be added) and this solution was used directly.

### 2.3 Coupling of Both the UV-Cleavable Ferrocene Molecule and the 3299 Antibody to the Latex

[symbol] Reaction carried out in the dark in order to avoid any contact with UV.

### 2.3.1 Coupling of the 3299 Antibody Followed by the UV-Cleavable Ferrocene Molecule [3299 Refers to the Clone Number for Anti-Alpha hCG for Detection of the Pregnancy Hormone hCG (Human Chorionic Gonadotrophin)]

A suspension of amidodextran-latex (1 ml, prepared according to section 2.2.1) was spun (15,500 rpm, 15° C.) for 20 min. The supernatant was discarded, the pellet was re-suspended in 900 μl of PBS (pH 7.0) using a bench vortex and an ultrasonic bath. 5 mg of the GMBS crosslinker in solution in 100 μl of DMF was added to the latex and the suspension was incubated for 45 min at room temperature with stirring (end-over-end mixer). The suspension was then spun (15,500 rpm, 15° C., 20 min). The supernatant was discarded, the pellet re-suspended in 1 ml of PBS (pH 7.0) and sonicated. The suspension was spun (15,500 rpm, 15° C., 20 min). The pellet was then re-suspended in 858 μl of PBS (pH 7.0) using a bench vortex and an ultrasonic bath. 142 μl of the modified 3299 antibody (prepared as described below in section 2.3.3) was then added and the latex was incubated 1 h30 at room temperature with stirring (end-over-end mixer). The suspension was then spun (15,500 rpm, 15° C., 20 min). The supernatant was discarded, the pellet was re-suspended in 1 ml of PBS (pH 7.0), sonicated. The suspension was spun (15,500 rpm, 15° C., 20 min).

The supernatant was discarded and the pellet was re-suspended in 325 μl of PBS (pH 7.0) using a bench vortex and an ultrasonic bath. 175 μl of DMF was then added (agitation) followed by 500 μl of a solution of the deprotected UV-cleavable ferrocene molecule (the deprotection of the UV-cleavable ferrocene molecule was performed as described in section 2.2.2.2). After sonication, the suspension was incubated overnight at room temperature with stirring (end-over-end mixer). The suspension was then spun (15,500 rpm, 15° C., 20 min). The supernatant was discarded, 1 ml of a solution of 35% DMP in PBS was added, the pellet was re-suspended (sonication). After agitation for 30 min at room temperature, the suspension was spun (15,500 rpm, 15° C.) for 20 min. The supernatant was discarded. This washing step was repeated 2 more times. The pellet was then re-suspended in a solution of 20% DMF in PBS using a bench vortex and an ultrasonic bath. After agitation for 30 min at room temperature, the suspension was spun (15,500 rpm, 15° C., 20 min). The supernatant was discarded. This washing step was repeated 1 more time. The pellet was then re-suspended in 1 ml of PBS, sonicated and the suspension was spun (15,500 rpm, 15° C., 20 min). The supernatant was discarded. Finally the pellet was re-suspended in 1 ml of PBS, sonicated and stored in the dark at 4° C.

### 2.3.2 Coupling of the UV-Cleavable Ferrocene Molecule Followed by 3299 Antibody

1 ml of a suspension of aminodextran-latex (prepared according to section 2.2.1) was spun (15,500 rpm, 15° C., 20 min). The supernatant was discarded, the pellet was re-suspended in 900 μl of PBS (pH 7.0) using a bench vortex and an ultrasonic bath. 5 mg of the GMBS crosslinker in solution in 100 μl of DMF was added to the latex and the suspension was incubated for 45 min at room temperature with stirring (end-over-end mixer). The suspension was then spun (15,500 rpm, 15° C., 20 min). The supernatant was discarded, the pellet re-suspended in 1 ml of PBS (pH 7.0) and sonicated. The suspension was spun (5,500 rpm, 15° C., 20 min). The supernatant was discarded, the pellet was then re-suspended in 325 μl of PBS (pH 7.0) using a bench vortex and an ultrasonic bath. 175 μl of DMF was then added (agitation) followed by 500 μof a solution of the deprotected UV-cleavable ferrocene linker (4.68*10⁻⁶ mol based on 2) (the deprotection of the UV-cleavable ferrocene 9 was performed as described in section 2.2.2.2). After sonication, the latex was stirred at room temperature for 5 min. 44 μl of a solution of Thiol-dPEG4-acid (3.54*10⁻² mol/l) was then added and the suspension was incubated overnight at room temperature with stirring (end over mixer). The suspension was then spun (15,500 rpm, 15° C., 20 min). The supernatant was discarded, 1 ml of a solution of 35% DMF in PBS was added, the pellet was re-suspended using a bench vortex and an ultrasonic bath. After agitation for 30 min at room temperature, the suspension was spun (15,500 rpm, 15° C.) for 20 min. The supernatant was discarded. This washing step was repeated 2 more times. The pellet was then re-suspended in a solution of 20% DMF in PBS and sonicated. After agitation for 30 min at room temperature, the suspension was spun (15,500 rpm, 15° C., 20 min). The supernatant was discarded. This washing step was repeated 1 more time. The pellet was then re-suspended in 1 ml of PBS, sonicated and the suspension was spun (15,500 rpm, 15° C., 20 min). The supernatant was discarded, the pellet was re-suspended (sonication) in 500 μl of MES (50 mM, pH 6.0).

5 mg of EDCI in solution in 150 μl of MES and 2 mg of NHS in solution in 150 μl of MES were added to the suspension while stirring. After 5 min of agitation (end-over-end mixer) at room temperature, a solution of 2 mg of amino dextran in 200 μl of MES was then added. The latex was incubated overnight at room temperature with stirring. The suspension was then spun (15,500 rpm, 15° C., 20 min). The supernatant was discarded, the pellet re-suspended in 1 ml of MES using a bench vortex and an ultrasonic bath. The suspension was spun (15,500 rpm, 15° C., 20 min). The supernatant was discarded and the pellet re-suspended (sonication) in 900 μl of PBS (pH 7.0).

5 mg of GMBS in solution in 100 g of DMF was then added to the suspension and then stirred for 45 min at room temperature. The suspension was then spun (15,500 rpm, 15° C., 20 min). The supernatant was discarded, the pellet re-suspended in 1 ml of PBS (pH 7.0) using a bench vortex and an ultrasonic bath. The suspension was spun (15,500 rpm, 15° C., 20 min). The supernatant was discarded and the pellet re-suspended (sonication) in 750 μl of PBS (pH 7.0).

250 μl of the modified 3299 antibody (prepared as described in section 2.3.3) was then added to the suspension and the latex was incubated overnight at room temperature with stirring (end over mixer). The suspension was then spun (15,500 rpm, 15° C., 20 min). The supernatant was discarded, the pellet re-suspended in 1 ml of PBS, sonicated. The suspension was then spun (15,500, 15° C., 20 min) and the supernatant was then discarded. This washing step was repeated 2 more times. Finally, pellet re-suspended (sonication) in 1 ml of PBS.

### 2.3.3 Preparation of 3299 Antibody

1 ml of 3299:4 antibody (3.41 mg/ml) was applied to a Nap 10 column equilibrated with 30 ml of PBS. 1.5 ml of PBS was then added to the column and collected.

The protein concentration was measured on a UV spectrophotometer at 280 nm: To 100 μl of the solution of antibody was added 900 μl of PBS. This 1 in 10 dilution gave an absorbance of 0.297→C=0.297*10 (dilution)/1.4=2.12 mg/ml

To 1.4 ml of the 3299 solution (2.97 mg, 1.98*10⁻⁸ mol) was added 15 μl of a solution of SAMSA in DMF at 8 mg/ml. The mixture was stirred overnight at room temperature.

To 400 μl of the 3299/SAMSA solution were added 35 μl of EDTA (0.1M) and 65 μl of hydroxylamine.HCl (1 M). The mixture was stirred for 10 min at room temperature and then applied to a Nap 5 column equilibrated with 15 ml of PBS. 1 ml of PBS was then added to the column and collected. The deprotected antibody can't be store and need to be used immediately.

### 2.4 Capture Phase

### 2.4.1 Synthesis of F108-PMPI

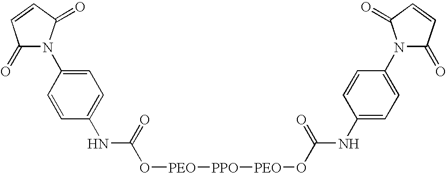

To a solution of F108 (1.13 g, 7.80*10⁻⁵ mol) in 10 ml of dry benzene was added PMPI (50 mg, 2.34*10⁻⁴ mol). The solution was stirred at room temperature overnight. The solution was then poured into 600 ml of diethyl ether, while stirring. The precipitate was collected by filtration and dried under vacuum. The solid was then dissolved in 8 ml of dry benzene, and precipitated in diethyl ether 2 more times. The product was then dried under high vacuum and stored under nitrogen at 4° C.

2.4.2 Coupling of 3468 Antibody to the Latex-Theoretical Latex Concentration 1% Solids (the 3468 Antibody Refers to an Anti-Beta hCG).

To 100 μl of 20 μm particles based on polystyrene (10% solids) was added 900 μl of deionised water (latex now at 1% solids). The suspension was then spun (13,500 rpm, 15° C.) for 10 min. The supernatant was discarded and the pellet re-suspended in 1 ml of deionised water using a bench vortex and an ultrasonic bath. This washing step was repeated 2 more times. The pellet was then re-suspended in 500 μl of deionised water. F108-PMPI (5 mg in 500 μl of deionised water) was added and the suspension was stirred (end-over-end mixer) at room temperature for 45 min. The suspension was then spun (13,500 rpm, 15° C., 10 min). The supernatant was discarded and the pellet re-suspended in 1 ml of deionised water. The suspension was spun (13,500 rpm, 15° C., 10 min). The supernatant was discarded and the pellet re-suspended in 500 μl of PBS (pH 7.0). 500 μl of the modified 3468 antibody (prepared as described below in section 2.4.3) was then added and the latex was incubated overnight at room temperature with stirring. The suspension was then spun (13,500 rpm, 15° C., 10 min). The supernatant was discarded, the pellet was re-suspended in 1 ml of PBS using a bench vortex and an ultrasonic bath. The suspension was spun (13,500 rpm, 15° C., 10 min). The supernatant was discarded. This washing step was repeated two more times. Finally, the pellet re-suspended in 1 ml of PBS and stored at 4° C.

### 2.4.3 Preparation of the Modified 3468 Antibody

1 ml of 3468 antibody (2.1 mg/ml) was applied to a Nap 10 column equilibrated with 30 ml of PBS (pH 7.0). 1.5 ml of PBS (pH 7.0) was then added to the column and collected.

The protein concentration was measured on a UV spectrophotometer at 280 nm: To 100 μl of the solution of antibody was added 900 μl of PBS (pH 7.0). This 1 in 10 dilution gave an absorbance of 0.221→C=0.221*10 (dilution)/1.4=1.578 mg/ml To 1.4 ml of the 3468 solution (2.21 mg, 1.47*10⁻⁸ mol) was added 12 μl of a solution of SAMSA in DMF at 8 mg/ml. The mixture was stirred overnight at room temperature.

To 900 μl of the 3468/SAMSA solution were added 35 μl of EDTA (0.1M) and 65 μl of hydroxylamine.HCl (1M). The mixture was stirred for 10 min at room temperature and then applied to a Nap 10 column equilibrated with 30 ml of PBS. 1.5 ml of PBS was then added to the column and collected. The deprotected antibody can't be stored and needs to be used immediately.

### 2.5 Photolysis

### 2.5.1 Photolysis of the UV-Cleavable Ferrocene Molecule in Solution

To a solution of UV-cleavable ferrocene 9 (0.7 mg, 1.09*10⁻⁶ mol) dissolved in 250 μl of methanol was added 250 μl of PBS. 30 μl of this solution was irradiated using a UV model B100A with a wavelength of 365 nm and an intensity of 8,900 μW/cm² at 10″. The UV was applied at approximately 15 cm from the solution.

The cleavage was followed by TLC every two minutes. Eluent used: Ethyl acetate/hexane (80/20) and DCM/MeOH/Et₃N (85/10/5).

2.5.2 Photocleavage of the UV-Cleavable Ferrocene Molecule from the Latex Particles

The irradiation was carried out for 5 min on variable bead concentrations using a UV lamp model B100A with a wavelength of 365 ml and an intensity of 8,900 μW/cm² at 10″. The UV were applied at approximately 15 cm from the solution.
Bead concentrations: a) 501 of beads (0.3% solids in theory)+10 μl of PBS b) 25 μl of beads (0.3% solids in theory)+25 μl of PBS c) 10 μl of beads (0.3% solids in theory)+40 μl of PBS
   Cyclic voltamograms were performed for each solution before and after irradiation by applying 17 μl of the solution to screen printed electrode (carbon working and counter electrode and a silver/silver chloride reference electrode).

### 2.6 Methodology for Section 3.3

17 μL of 400 nm (% solids) TRL particles sensitised with UV cleavable ferrocene compound was added to a screen printed electrode (carbon working and counter electrodes and silver/silver chloride reference electrode). The solution covered the working, counter and reference electrode.

A chronoamperometry measurement was started as soon as the above solution was applied to the electrode and after approximately 50 seconds the UV source (Green LED, 360 nm wavelength) was turned on. The LED input voltage was 20 mV and the LED was positioned directly above the electrode.

This procedure was repeated for several different 400 nm bead concentrations, the concentrations used were 6.5E+09 2.74E+09, 1.37E+09, 6.85E+08, 3.43E+08, 1.71E+08, 8.56E+07, 4.28E+07 per ml.

A UV cleaved ferrocene molecule calibration curve was produced by performing identical measurements to above but with known concentrations of the UV cleaved ferrocene compound (pre-synthesised). The concentrations used were 100, 50, 25, 12.5, 6.25 and 3.125 μM.

The chronoamperometry measurement parameters were as follows.
First conditioning potential=0V
Equilibration time=4 seconds
Interval time=1 second
Number of potential step=1
0.42V potential
300 second duration

### 2.7 Methodology for Section 3.4

A thin layer cell/capillary fill device was constructed in the following fashion. A double sided adhesive tape (code 7840, adhesive research) was placed over a screen printed electrode (carbon working and counter electrodes and silver/silver chloride reference electrode) upon which a glass cover slip was placed creating a 90 μm capillary gap.

6 μL of sample solution (400 nm beads, PBS) was applied to the capillary fill device and a chronoamperometry measurement performed (identical procedure to exp 1). The LED input voltage was varied (22 and 38 mV).

### 2.8 Methodology for Section 3.8

50 μL (% solids) of 3468 (anti hCG)/UV cleavable ferrocene molecule sensitised 400 nm n beads were mixed with 50 μL of hCG standard (0 or 400 mIU) and 75 μL (% solids) of 20 μm beads sensitised with 3299 (anti hCG) antibody for 30 minutes (agitated on plate shaker).

A microfluidic device incorporating the immunofilter 3 (IMF3) device was constructed in the following fashion. Double sided adhesive tape (code 7840, adhesive research) was placed upon and around the filter region and the capillary channel of the IMF3 device. A screen printed electrode (polyester substrate, carbon working, reference and counter electrodes) was placed over the adhesive tape creating the microfluidic device.

20 μL of the incubated sample solution was applied to the microfluidic device and drawn through the device using a gel blot sink which was applied to the tail region of the actual immunofilter. 10 μL of PBS wash solution was then drawn through the device and finally 2.5 μL of “resuspension” PBS was added. Chronoamperometry measurements were performed, electrochemical conditions were as previously described. The LED input voltage was 22 mV.

1. A method of determining the presence or amount of analyte in a fluid sample, which comprises:
(a) contacting a fluid sample with a binding reagent that comprises a plurality of cleavable species and wherein said species, when cleaved, are detectable using electrochemical means;
(b) separating any binding reagent-analyte complex that forms from the unbound binding reagent;
(c) cleaving the cleavable species from the immobilized binding reagent-analyte complex; and
(d) detecting the cleaved species using electrochemical means.

2. A method according to claim 1, wherein the binding reagent-analyte complex is separated from the unbound binding reagent by immobilization of the binding reagent-analyte complex in a capture phase.

3. A method according to claim 1, wherein the binding reagent comprises at least 10⁶ cleavable species.

4. A method according to claim 1, wherein the cleavable species are photocleavable or acid cleavable.

5. A method according to claim 1, wherein the cleavable species show electrochemical activity when they have been cleaved from the binding reagent.

6. A method according to claim 1, wherein the cleavable species are transformable, after being cleaved from the binding reagent, into an electrochemically active species.

7. A method according to claim 1, wherein the cleavable species, after being cleaved from the binding reagent, result in further species becoming electrochemically active.

8. A method according to claim 1, wherein the cleavable species is not electrochemically active when attached to the binding reagent.

9. A method according to claim 1, wherein the cleavable species comprises a moiety derived from ferrocene, nitrophenol, aminophenol, hydroquinone, salicylic acid or sulfosalicylic acid.

10. A method according to claim 1, wherein the cleavable species comprises a moiety derived from ferrocene aldehyde.

11. A method according to claim 1, wherein the cleavable species comprises one or more moieties derived from ethylene glycol.

12. A method according to claim 1, wherein the binding reagent comprises a central core.

13. A method according to claim 12, wherein the central core is a polymer sphere.

14. A method according to claim 1, wherein the binding reagent comprises at least one dendritic or polymeric moiety.

15. A method according to claim 14 wherein the cleavable species are attached to the dendritic or polymeric moiety.

16. A method according to claim 15, wherein the dendritic or polymeric moiety is attached to the central core.

17. A method according to claim 14, wherein the dendritic or polymeric moiety is provided on the outer surface of the cleavable species.

18. A method according to claim 14, wherein the polymeric moiety is derived from dextran.

19. A method according to claim 1, wherein the electrochemical means comprises an electrode.

20. A binding reagent suitable for use in an immunoassay which comprises a plurality of cleavable species and wherein said species, when cleaved, are detectable using electrochemical means.

21. A method of performing an immunoassay which uses a binding regent which comprises a plurality of cleavable species and wherein said species, when cleaved, are detectable using electrochemical means.

22. An assay kit for measuring the amount or presence of an analyte in a sample, comprising:
a binding reagent which comprises a plurality of cleavable species and wherein said species, when cleaved, are detectable using electrochemical means;
a capture phase comprising a support having a reagent which is capable of binding or attaching to a binding-reagent-analyte complex; and
an electrode capable of detecting the cleavable species, when cleaved, to provide an indication of the presence or amount of analyte present.
